(19) United States
(12) Patent Application Publication
(10) Pub. No.: US 2010/0267719 A1
(43) Pub. Date: Oct. 21, 2010

(54) Enhanced Indolinone Based Protein Kinase Inhibitors

(75) Inventors: Congxin Liang, Jupiter, FL (US); Yangbo Feng, Palm Beach Gardens, FL (US); Tomas Vojkovsky, Palm Beach Gardens, FL (US)

Correspondence Address: SCHWEGMAN, LUNDBERG & WOESSNER, P.A., P.O. BOX 2938, MINNEAPOLIS, MN, 55402, US

(73) Assignee: The Scripps Research Institute, La Jolla, CA (US)

(21) Appl. No.: 11/920,583

(22) PCT Filed: May 26, 2006
(86) PCT No.: PCT/US2006/020363

§ 371 Date: Sep. 24, 2009

Related U.S. Application Data
(60) Provisional application No. 60/685,144, filed on May 26, 2005.
(60) Provisional application No. 60/754,360, filed on Dec. 28, 2005.

(51) Int. Cl. A61K 31/5377; C07D 403/06; A61K 31/405; C07D 413/14; A61P 29/00; A61P 35/00

(52) U.S. Cl. 514/235.2; 548/468; 514/414; 544/144

(57) ABSTRACT

Alpha-hydroxy-omega-(2-oxo-indolylidenemethyl-pyrrole-3′-carbonyl)amino alkanoic acid and amide derivatives have enhanced and unexpected drug properties as inhibitors of protein kinases and are useful in treating disorders related to abnormal protein kinase activities such as cancer.

### FIELD OF INVENTION

The invention relates to protein kinase inhibitors and to their use in treating disorders related to abnormal protein kinase activities such as cancer and inflammation. More particularly, the invention relates to alpha-hydroxy-ω-(2-oxo-indolylidenemethyl-pyrrole-3′-carbonyl)amino alkanoic acid and amide derivatives and their pharmaceutically acceptable salts employable as protein kinase inhibitors.

### BACKGROUND

Protein kinases are enzymes that catalyze the phosphorylation of hydroxyl groups of tyrosine, serine, and threonine residues of proteins. Many aspects of cell life (for example, cell growth, differentiation, proliferation, cell cycle and survival) depend on protein kinase activities. Furthermore, abnormal protein kinase activity has been related to a host of disorders such as cancer and inflammation. Therefore, considerable effort has been directed to identifying ways to modulate protein kinase activities. In particular, many attempts have been made to identify small molecules that act as protein kinase inhibitors.

Several pyrrolyl-indolinone derivatives have demonstrated excellent activity as inhibitors of protein kinases (Ladd et al. FASEB J. 16, 681, 2002; Smolich et al. Blood, 97, 1413, 2001; Mendel et al. Clinical Cancer Res. 9, 327, 2003; Sun et al. J. Med. Chem. 46, 1116, 2003). The clinical utility of these compounds has been promising, but has been partially compromised due to the relatively poor aqueous solubility and/or other drug properties. What is needed is a class of modified pyrrolyl-indolinone derivatives having both inhibitory activity and enhanced drug properties.

### SUMMARY

The invention is directed to alpha-hydroxy-omega-(2-oxo-indolylidenemethyl-pyrrole-3′-carbonyl)amino alkanoic acid and amide derivatives and to their use as inhibitors of protein kinases. It is disclosed herein that alpha-hydroxy-ω-(2-oxo-indolylidenemethyl-pyrrole-3′-carbonyl) amino alkanoic acid and amide derivatives have enhanced and unexpected drug properties that advantageously distinguish this class of compounds over known pyrrolyl-indolinone derivatives having protein kinase inhibition activity and over their corresponding beta-hydroxy-ω-(2-oxo-indolylidenemethyl-pyrrole-3′-carbonyl)amino alkanoic acid and amide derivatives. It is also disclosed herein that alpha-hydroxy-ω-(2-oxo-indolylidenemethyl-pyrrole-3′-carbonyl)amino alkanoic acid and amide derivatives are useful in treating disorders related to abnormal protein kinase activities such as cancer.

One aspect of the invention is directed to a compound represented by Formula (I):

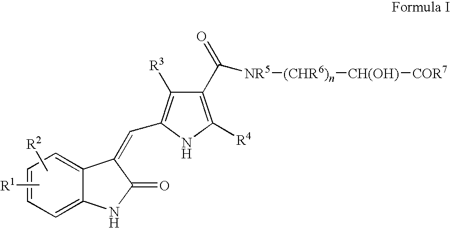

In Formula (I), R¹ is selected from the group consisting of hydrogen, halo, (C1-C6) alkyl, (C3-C8) cycloalkyl, (C1-C6) haloalkyl, hydroxy, (C1-C6) alkoxy, amino, (C1-C6) alkylamino, amide, sulfonamide, cyano, substituted or unsubstituted (C6-C10) aryl; R² is selected from the group consisting of hydrogen, halo, (C1-C6) alkyl, (C3-C8) cycloalkyl, (C1-C6) haloalkyl, hydroxy, (C1-C6) alkoxy, (C2-C8) alkoxyalkyl, amino, (C1-C6) alkylamino, (C6-C10) arylamino; R³ is selected from the group consisting of hydrogen, (C1-C6) alkyl, (C6-C10) aryl, (C5-C10) heteroaryl, and amide; R⁴, R⁵ and R⁶ are independently selected from the group consisting of hydrogen and (C1-C6) alkyl; R⁷ is selected from the group consisting of hydroxy, (C1-C6) O-alkyl, (C3-C8) O-cycloalkyl, and NR⁸R⁹; where R⁸ and R⁹ are independently selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C₁-C₆) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids; and n is 1, 2, or 3. Alternatively, this aspect of the invention may be directed to a pharmaceutically acceptable salt, its tautomer, a pharmaceutically acceptable salt of its tautomer, or a prodrug of the compound of Formula (I). Preferred species of the invention include compounds represented by the following structures:

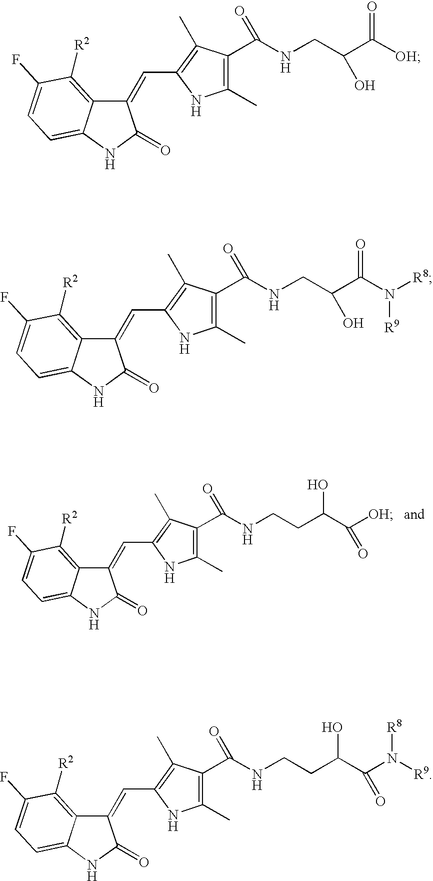

In the above structures, R² is selected from the group consisting of hydrogen and fluoro. More particularly, a preferred stereoisomer is represented by the following structure:

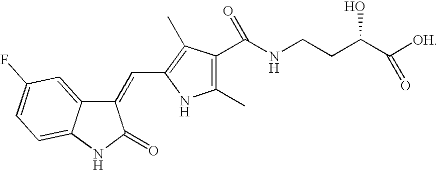

A first subgenus of this aspect of the invention is represented by Formula (II):

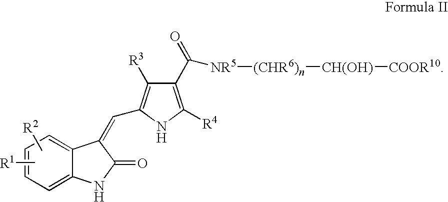

In Formula (II), R¹⁹ is selected from the group consisting of hydrogen, (C1-C6) alkyl, and (C3-C8) cycloalkyl. In preferred species of this first subgenus, R¹ and R² are independently selected from the group consisting of hydrogen and fluoro; R³ and R⁴ are methyl; R⁵, R⁶, and R¹⁰ are hydrogen; and n is 1 or 2. Preferred species are represented by the following compounds:

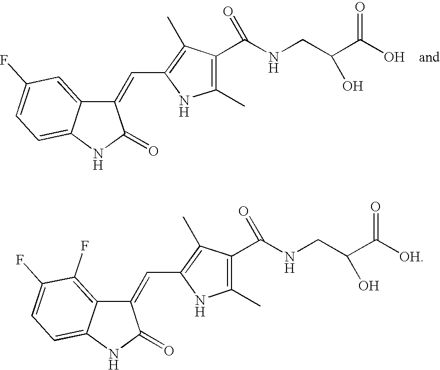

A preferred chiral species is represented by the following compound:

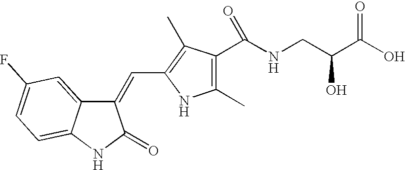

A second subgenus of this aspect of the invention is directed to a compound according to Formula (III) or a salt, tautomer, or prodrug thereof:

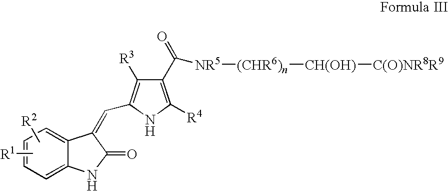

In preferred species of this second subgenus, R¹ and R² are independently selected from the group consisting of hydrogen, halo, cyano; R³, R⁴, R⁵ and R⁶ are independently hydrogen or (C1-C6))alkyl; n is 1 or 2; and R⁸ and R⁹ are selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids. Preferred species of the second subgenus are represented by the following structures:

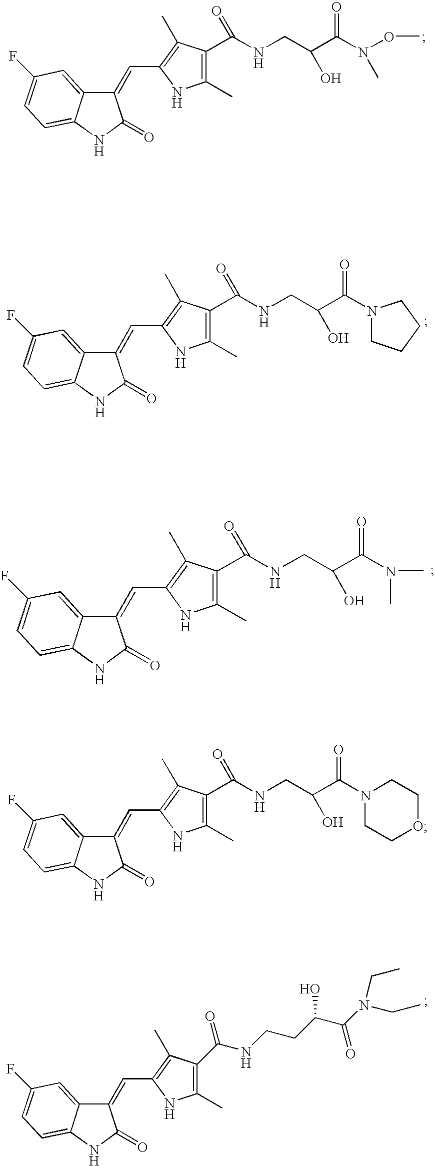

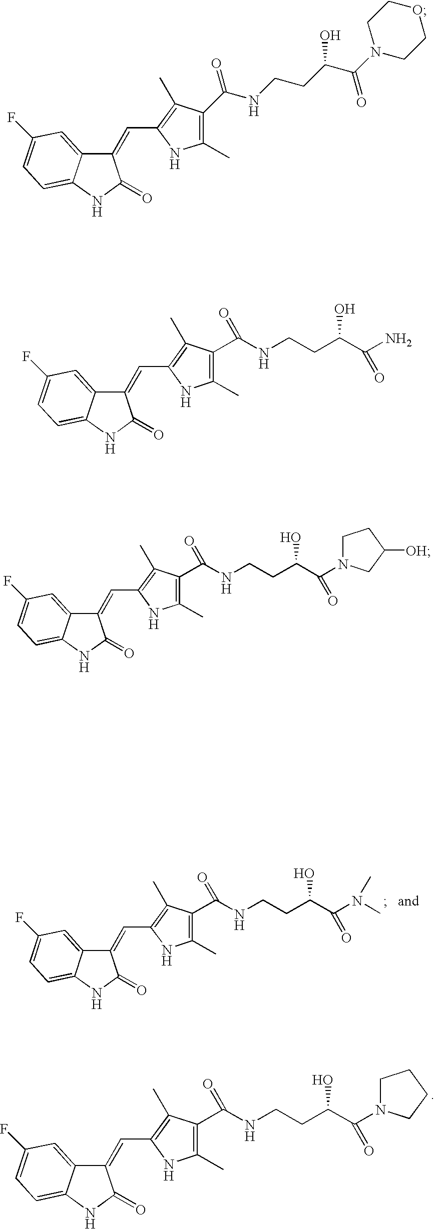

In a first subset of the second subgenus, n is 1. Preferred species within this first subset are represented by the following structures:

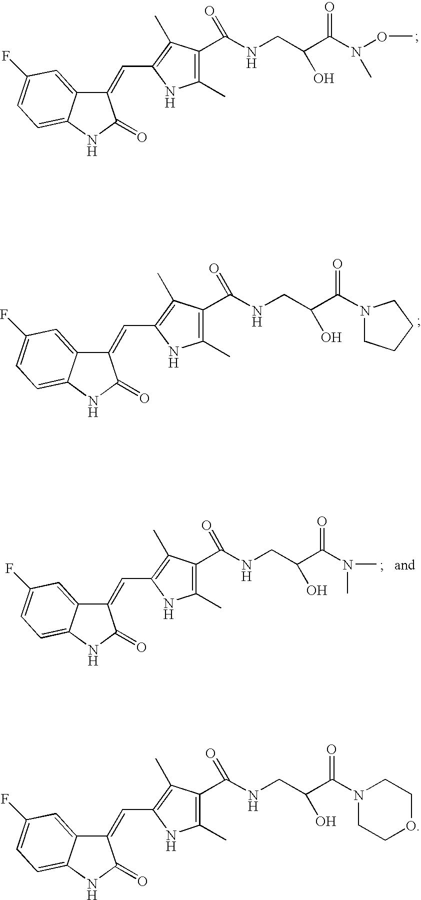

Preferred chiral species within the first subset of the second subgenus are represented by the following structures:

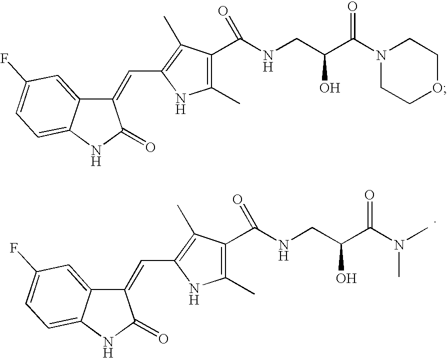

Further preferred chiral species within the first subset of the second subgenus are represented by the following structures:

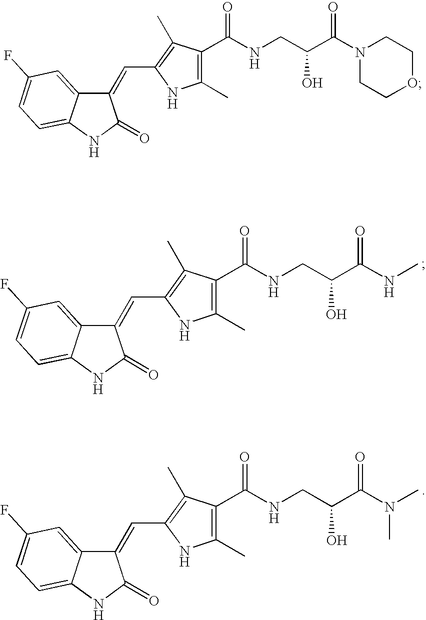

In a second subset of the second subgenus, n is 2. Preferred species within this first subset are represented by the following structures:

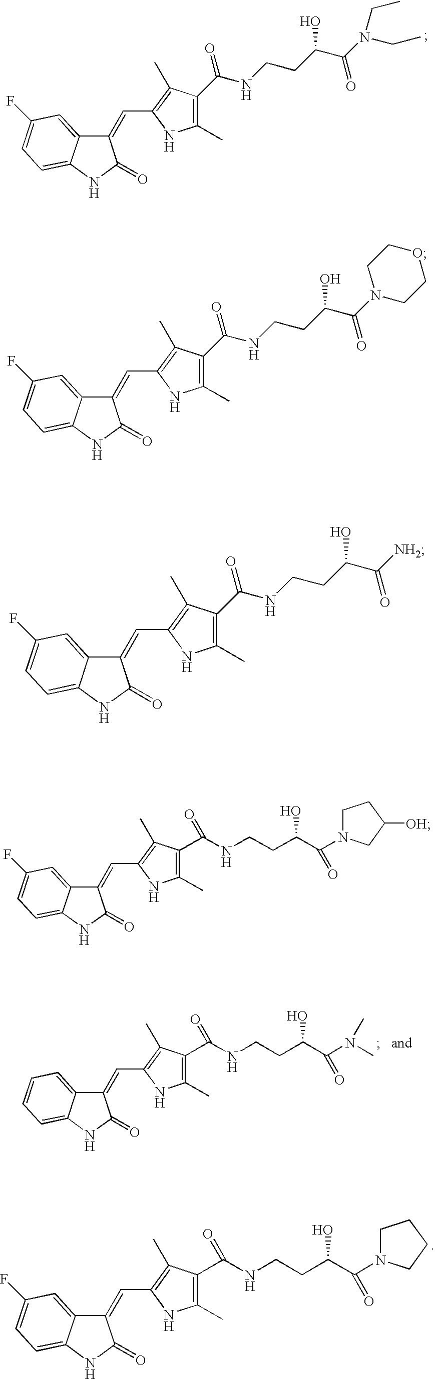

Further preferred species of the first aspect of the invention are represented by the following structures:

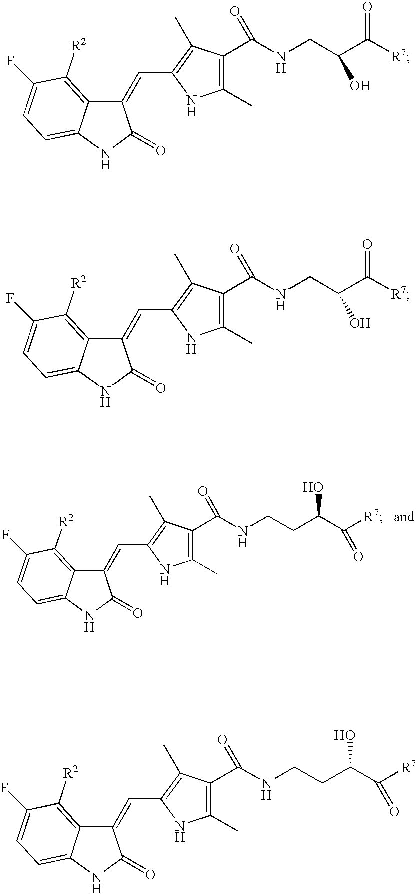

In the above structures, R² is selected from the group consisting of hydrogen and fluoro; and R⁷ is selected from the group consisting of hydroxyl or radicals represented by the following structures:

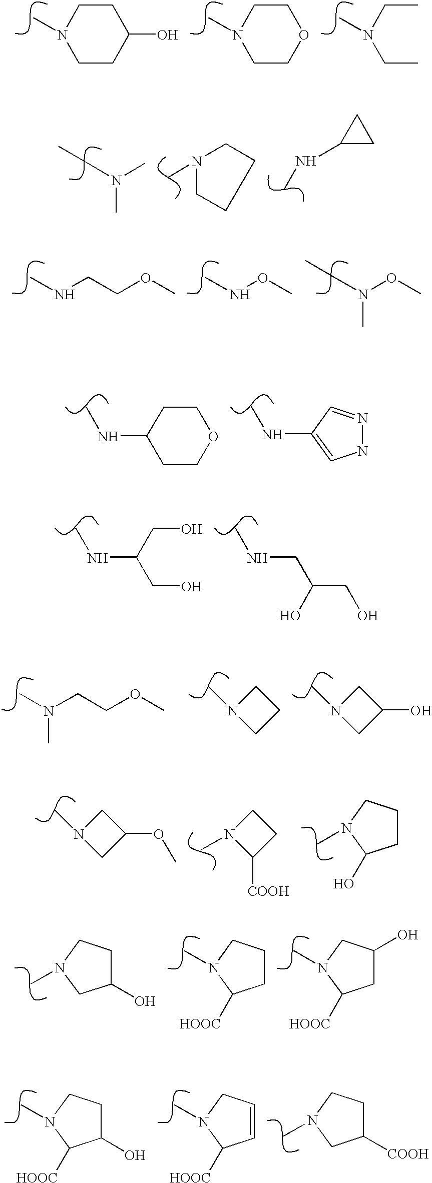

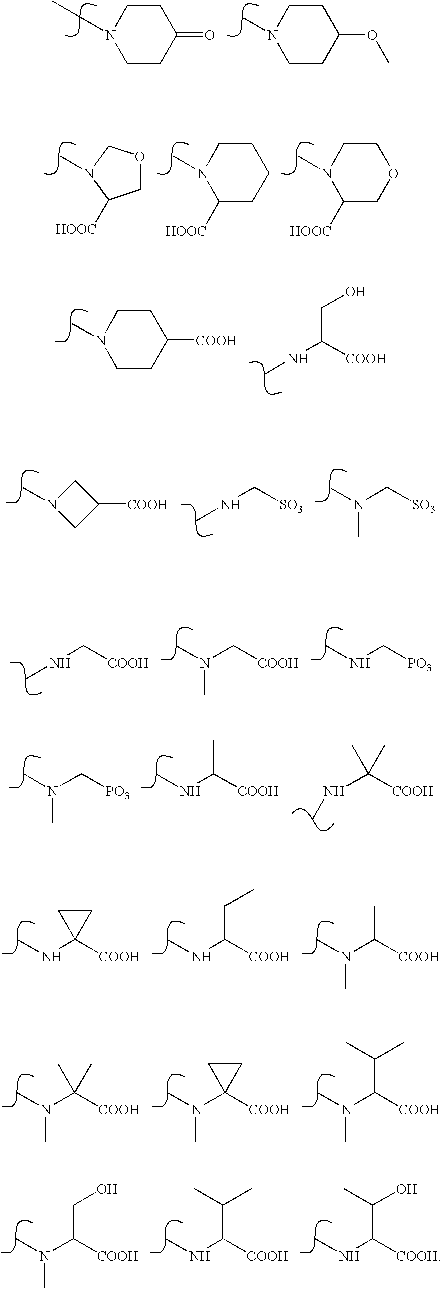

A second aspect of the invention is directed to a method for the modulation of the catalytic activity of a protein kinase with a compound or salt represented by Formulas I-III, above. In a preferred mode of the second aspect of the invention, said protein kinase is selected from the group of receptors consisting of VEGF, PDGF, c-kit, Flt-3, Axl, and TrkA.

### Utility:

The present invention provides compounds capable of regulating and/or modulating protein kinase activities of, but not limited to, VEGFR and/or PDGFR. Thus, the present invention provides a therapeutic approach to the treatment of disorders related to the abnormal functioning of these kinases. Such disorders include, but not limited to, solid tumors such as glioblastoma, melanoma, and Kaposi's sarcoma, and ovarian, lung, prostate, pancreatic, colon and epidermoid carcinoma. In addition, VEGFR/PDGFR inhibitors may also be used in the treatment of restenosis and diabetic retinopathy.

Furthermore, this invention relates to the inhibition of vasculogenesis and angiogenesis by receptor-mediated pathways, including the pathways comprising VEGF receptors, and/or PDGF receptors. Thus the present invention provides therapeutic approaches to the treatment of cancer and other diseases which involve the uncontrolled formation of blood vessels.

### BRIEF DESCRIPTION OF DRAWINGS

FIG. 1 illustrates a scheme showing the synthesis of the acid 1-3 and the corresponding amides, 1-4. The starting carboxylic acid is synthesized according to the supplemental material of Sun, L.; et al., J. Med. Chem. 2003, 46, 1116-1119.

FIG. 2 illustrates a scheme showing the synthesis of the amide series, 2-3.

FIG. 3 shows example compounds and some of their activities against KDR.

FIG. 4 shows additional compounds that were tested for activity.

### EXAMPLES

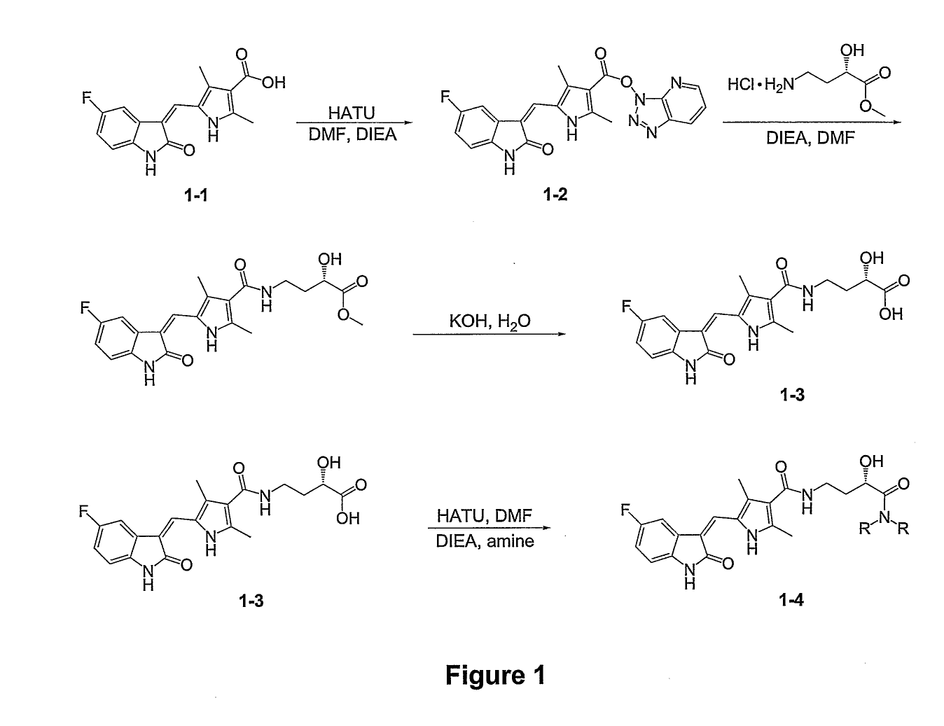

Examples 1-7: The Synthesis of Acid (1-3) and Amides (1-4) is Shown in FIG. 1.

### Example 1

### (S)-4-({5-[5-Fluoro-2-oxo-1,2-dihydro-indol-(3Z)-ylidenemethyl]-2,4-dimethyl-1H-pyrrole-3-carbonyl}-amino)-2-hydroxy-butyric acid:

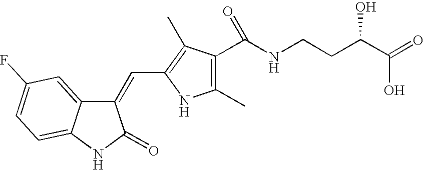

Compound 1-1 was prepared by following a literature procedure used for similar compounds (Li Sun, Chris Liang, et al; Discovery of 5-[5-Fluoro-2-oxo-1,2-dihydroindol-(3Z)-ylidenemethyl]-2,4-dimethyl-1H-pyrrole-3-carboxylic Acid (2-Diethylaminoethyl)amide, a Novel Tyrosine Kinase Inhibitor Targeting Vascular Endothelial and Platelet-Derived Growth Factor Receptor Tyrosine Kinase. J. Med. Chem. 2003, 46, 1116-1119). Compound 1-1 and DIEA (di-isopropyl ethylamine) (3 equiv) were suspended in dry DMF at room temperature (FIG. 1). After sonication (5 min), HATU (0.99 equiv) was added. The suspension became a clear solution after stirring approximately 1 minute at room temperature. Precipitation was observed after another 15 min. After DMF was removed under reduced pressure, anhydrous acetonitrile was added. The precipitate was collected by filtration, washed several times using acetonitrile, and dried under high vacuum for 2 days to give compound 1-2. LC-MS and NMR spectroscopy confirmed the structure of 1-2. To a solution of compound 1-2 (1.27 mmol) and DIEA (3 equiv) in DMF, the hydrogen chloride salt of methyl (2S)-4-amino-2-hydroxybutyrate (1.5 equiv, prepared earlier by refluxing the free amino acid (Aldrich) in dry methanol with 1.2 equiv HCl) was added. After stirring at 25° C. for 2 h (at which time LC-MS showed the completion of the reaction), KOH in water (5 equiv) was added, and stirring was continued until the hydrolysis was complete (monitored by LC-MS). The solvents were removed by evaporation under reduced pressure. Aqueous HCl (1N) was added to the residue, and the precipitate was collected by filtration, washed with water, and dried under high vacuum to obtain the title compound (0.5 g, 98%). LC-MS: single peak at 254 nm, MH⁺ calcd. for C₂₀H₂₀FN₃O₅: 402, obtained: 402.

### Example 2-7

The general procedure for the synthesis of amides of Example 1: An amine (2 equiv) was added to a solution of the acid from Example 1, HATU (1.05 mmol), and DIEA (5 equiv) in DMF (5 mL). After the solution was stirred at 25° C. for 2 h, aqueous HCl (2 mL, 1N) was added. This solution was subjected to preparative HPLC to obtain the pure amide product, which was subsequently characterized by LC-MS and NMR spectroscopy.

### Example 2

### 5-[5-Fluoro-2-oxo-1,2-dihydro-indol-(3Z)-ylidenemethyl]-2,4-dimethyl-1H-pyrrole-3-carboxylic acid ((S)-3-hydroxy-4-oxo-4-pyrrolidin-1-yl-butyl)-amide

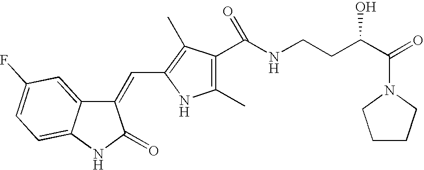

Preparative HPLC gave 32 mg of the title compound (34%) from 90 mg starting material (acid). LC-MS: single peak at 254 nm, MH⁺ calcd. for C₂₄H₂₇FN₄O₄: 455, obtained: 455.

### Example 3

### 5-[5-Fluoro-2-oxo-1,2-dihydro-indol-(3Z)-ylidenemethyl]-2,4-dimethyl-1H-pyrrole-3-carboxylic acid [(S)-3-hydroxy-4-((R)-3-hydroxypyrrolidin-1-yl)-4-oxo-butyl]-amide

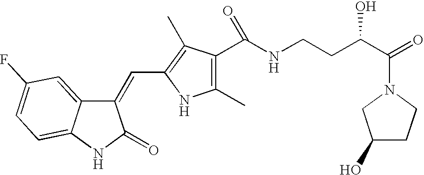

Preparative HPLC gave 27 mg of the title compound (41%) from 61 mg starting material (acid). LC-MS: single peak at 254 nm, MH⁺ calcd. for C₂₄H₂₇FN₄O₅: 471, obtained: 471.

### Example 4

### 5-[5-Fluoro-2-oxo-1,2-dihydro-indol-(3Z)-ylidenemethyl]-2,4-dimethyl-1H-pyrrole-3-carboxylic acid ((S)-3-dimethylcarbamoyl-3-hydroxy-propyl)-amide

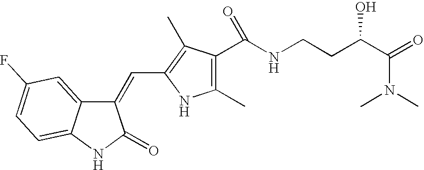

Preparative HPLC gave 22 mg of the title compound (37%) from 61 mg starting material (acid). LC-MS: single peak at 254 nm, MH⁺ calcd. for C₂₂H₂₅FN₄O₄: 429, obtained: 429.

### Example 5

### 5-[5-Fluoro-2-oxo-1,2-dihydro-indol-(3Z)-ylidenemethyl]-2,4-dimethyl-1H-pyrrole-3-carboxylic acid ((S)-3-di-ethylcarbamoyl-3-hydroxy-propyl)-amide

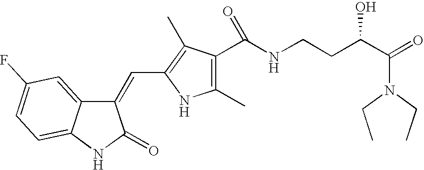

Preparative HPLC gave 43 mg of the title compound (27%) from 140 mg starting material (acid). LC-MS: single peak at 254 nm, MH⁺ calcd. for C₂₄H₂₉FN₄O₄: 457, obtained: 457.

### Example 6

### 5-[5-Fluoro-2-oxo-1,2-dihydro-indol-(3Z)-ylidenemethyl]-2,4-dimethyl-1H-pyrrole-3-carboxylic acid ((S)-3-carbamoyl-3-hydroxy-propyl)-amide

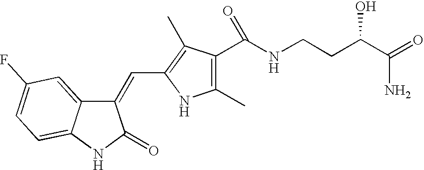

Preparative HPLC gave 15 mg of the title compound (20%) from 81 mg starting material (acid). LC-MS: single peak at 254 nm, MH⁺ calcd. for C₂₀H₂₁FN₄O₄: 401, obtained: 401.

### Example 7

### 5-[5-Fluoro-2-oxo-1,2-dihydro-indol-(3Z)-ylidenemethyl]-2,4-dimethyl-1H-pyrrole-3-carboxylic acid ((S)-3-hydroxy-4-morpholin-4-yl-4-oxo-butyl)-amide

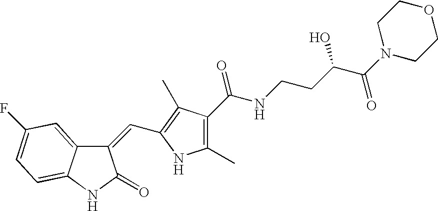

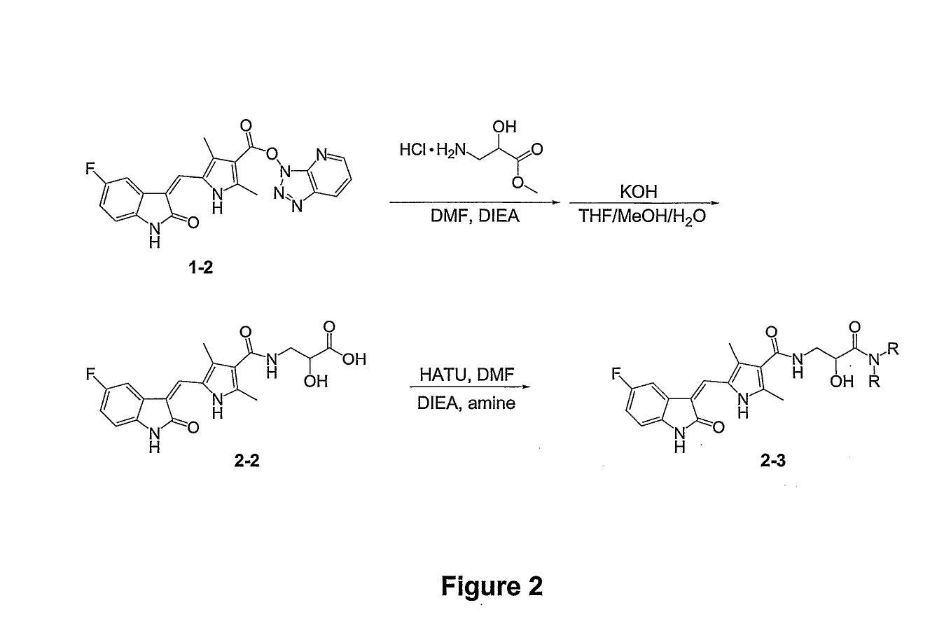

Preparative HPLC gave 18 mg of the title compound (21%) from 81 mg starting material (acid). LC-MS: single peak at 254 nm, MH⁺ calcd. for C₂₄H₂₇FN₄O₅: 471, obtained: 471.
Examples 8-11: The synthesis of acid (2-2) and amides (2-3) is shown in FIG. 2.

### Example 8

### 3-({5-[5-Fluoro-2-oxo-1,2-dihydro-indol-(3Z)-ylidenemethyl]-2,4-dimethyl-1H-pyrrole-3-carbonyl}-amino)-2-hydroxy-propionic acid

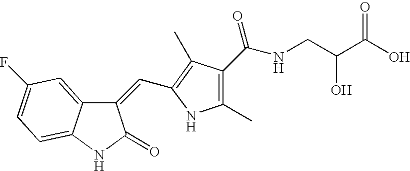

To a solution of compound 1-2 (1.0 mmol) and DIEA (3 equiv) in DMF, the HCl salt of methyl 3-amino-2-hydroxypropionate (1.2 equiv, prepared by refluxing the isoserine in dry methanol with 1.2 equiv HCl) was added. After stirring at 25° C. for 2 h (at which time LC-MS showed the completion of the reaction), KOH in water (5 equiv) was added, and the stirring was continued until the hydrolysis was complete (monitored by LC-MS). The solvents were removed by evaporation under reduced pressure. Aqueous HCl (1N) was added to the residue, and the precipitate was collected by filtration, washed with water, and dried under high vacuum to obtain compound 2-2 (0.33 g, 85%). LC-MS: single peak at 254 nm, MH⁺ calcd. for C₁₆H₁₈FN₃O₆: 388, obtained: 388.

Examples 9-11: The general procedure for the synthesis of amides of Example 8: An amine (2 equiv) was added to a solution of the acid, HATU (1.05 mmol), and DIEA (5 equiv) in DMF (5 mL). After the solution was stirred at 25° C. for 2 h, aqueous HCl (2 mL, 1N) was added. This solution was subjected to preparative HPLC to obtain the pure amide product, which was subsequently characterized by LC-MS and NMR spectroscopy.

### Example 9

### 5-[5-Fluoro-2-oxo-1,2-dihydro-indol-(3Z)-ylidenemethyl]-2,4-dimethyl-1H-pyrrole-3-carboxylic acid (2-dimethylcarbamoyl-2-hydroxy-ethyl)-amide

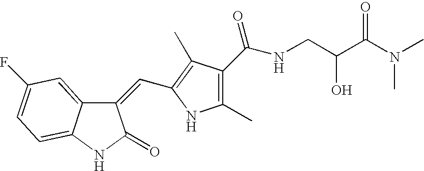

Preparative HPLC gave 50 mg of the title compound (72%) from 65 mg starting material (acid). LC-MS: single peak at 254 nm, MH⁺ calcd. for C₂₁H₂₃FN₄O₄: 415, obtained: 415. ¹H NMR (DMSO-d₆, 400 MHz) δ 13.67 (s, 1H), 10.87 (s, 1H), 7.75 (dd, J=2.4 Hz, 9.6 Hz, 1H), 7.70 (s, 1H), 7.56 (t, J=6.0 Hz, 1H), 6.92 (m, 1H), 6.83 (dd, J=4.8 Hz, 8.4 Hz, 1H), 4.53 (t, J=5.6 Hz, 1H), 3.48-3.25 (m, 2H), 3.08 (s, 3H), 2.85 (s, 3H), 2.43 (s, 3H), 2.41 (s, 3H).

### Example 10

### 5-[5-Fluoro-2-oxo-1,2-dihydro-indol-(3Z)-ylidenemethyl]-2,4-dimethyl-1H-pyrrole-3-carboxylic acid (2-hydroxy-3-(morpholin-4-yl)-3-oxo-propyl)-amide

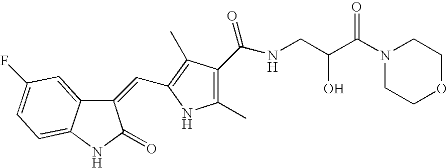

Preparative HPLC gave 14 mg of the title compound (18%) from 65 mg starting material (acid). LC-MS: single peak at 254 nm, MH⁺ calcd. for C₂₃H₂₅FN₄O₅: 457, obtained: 457. ¹H NMR (DMSO-d₆, 400 MHz) δ 13.68 (s, 1H), 10.90 (s, 1H), 7.75 (dd, J=2.4 Hz, 9.6 Hz, 1H), 7.71 (s, 1H), 7.60 (t, J=6.0 Hz, 1H), 6.92 (m, 1H), 6.83 (dd, J=4.4 Hz, 8.4 Hz, 1H), 5.2 (b, 1H), 4.51 (t, J=6.0 Hz, 1H), 3.65-3.35 (m, 10H), 2.43 (s, 3H), 2.41 (s, 3H).

### Example 11

### 5-[5-Fluoro-2-oxo-1,2-dihydro-indol-(3Z)-ylidenemethyl]-2,4-dimethyl-1H-pyrrole-3-carboxylic acid [2-hydroxy-2-(methoxy-methyl-carbamoyl)-ethyl]-amide

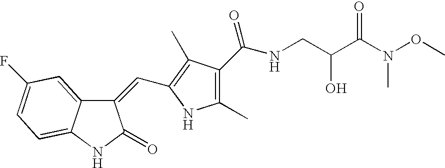

Preparative HPLC gave 16 mg of the title compound (18%) from 80 mg starting material (acid). LC-MS: single peak at 254 nm, MH⁺ calcd. for C₂₁H₂₃FN₄O₅: 431, obtained: 431. ¹H NMR (DMSO-d₆, 400 MHz) δ 13.67 (s, 1H), 10.89 (s, 1H), 7.75 (dd, J=2.0 Hz, 9.2 Hz, 1H), 7.70 (s, 1H), 7.55 (t, J=5.6 Hz, 1H), 6.92 (m, 1H), 6.82 (dd, J=4.8 Hz, 8.8 Hz, 1H), 4.51 (t, J=6.0 Hz, 1H), 3.74 (s, 3H), 3.55-3.40 (m, 2H), 3.13 (s, 3H), 2.42 (s, 3H), 2.41 (s, 3H).

The compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

### Exemplary Chiral Species

A general scheme for synthesizing chiral species of the invention is outline below:

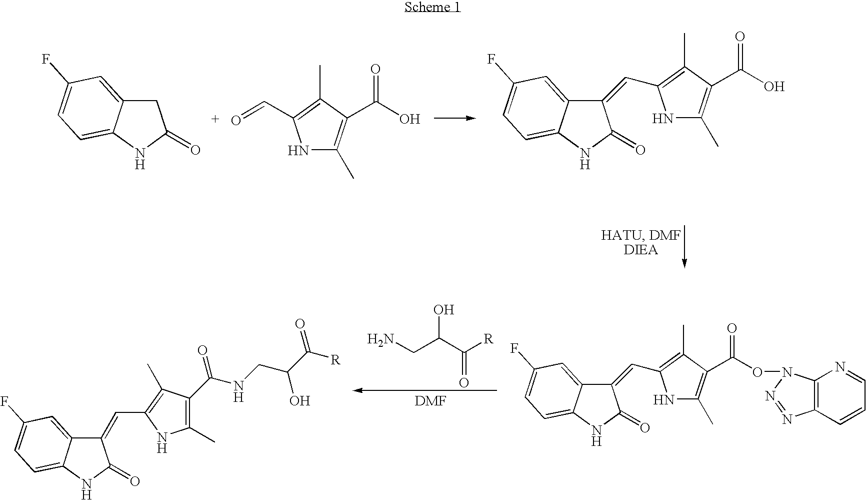

### Step 1:

A mixture of 5-fluoro-1,3-dihydroindol-2-one (1.62 g, 10.2 mmol), 5-formyl-2,4-dimethyl-1H-pyrrole-3-carboxylic acid (1.96 g, 10.7 mmol), pyrrolidine (12 drops) and absolute ethanol was heated to reflux for 3 hours. The mixture was cooled to 25° C. and the solids were collected by filtration. The solids were stirred with ethanol (30 mL) at 72° C. for 30 min. The mixture was cooled to 25° C. and the solids were collected again by filtration, washed with ethanol (6 mL), and dried under vacuum overnight to give an orange solid (Z)-5-((5-fluoro-2-oxoindolin-3-ylidene)methyl)-2,4-dimethyl-1H-pyrrole-3-carboxylic acid (3.094 g, 96%). LC-ESIMS observed [M+H]⁺ 300.95 (calculated for C₁₆H₁₃FN₂O₃ 300.09).

### Step 2:

(Z)-5-(5-fluoro-2-oxoindolin-3-ylidene)methyl)-2,4-dimethyl-1H-pyrrole-3-carboxylic acid (3.094 g, 10.3 mmol) was suspended in DMF (15 mL), and stirred for 5 minutes. DIEA (2.7 mL, 15.5 mmol) was then added and the mixture was stirred for 10 minutes. HATU (3.91 g, 10.28 mmol) was added and the reaction mixture was stirred at 25° C. for completion. LC/MS detected the completion of the reaction. Most of the DMF was removed and the residue was suspended in ACN and stirred for another 40 minutes. The solid was collected by filtration, washed with ACN, and dried under high vacuum overnight. (Z)-3H-[1,2,3]triazolo[4,5-b]pyridin-3-yl 5-((5-fluoro-2-oxoindolin-3-ylidene)methyl)-2,4-dimethyl-1H-pyrrole-3-carboxylate (3.97 g, 92%) was obtained. LC-ESIMS observed [M+H]⁺ 418.68 (calculated for C₂₁H₁₅FN₆O₃ 418.12).

### Step 3:

To (Z)-3H-[1,2,3]triazolo[4,5-b]pyridin-3-yl 5-((5-fluoro-2-oxoindolin-3-ylidene)methyl)-2,4-dimethyl-1H-pyrrole-3-carboxylate (1.0 eq) DMF solution was added amine (1.2 eq), the reaction mixture was stirred at 25° C. for 2 h. LC/MS was applied to detect the completion of the reaction. Remove DMF under reduced pressure and the crude was precipitated with 5% diethylamine/methanol (3 mL) under sonication, the solid was collected by filtration and washed with 5% diethylamine/methanol (1 mL) twice.

### Example 12

### Synthesis of (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid

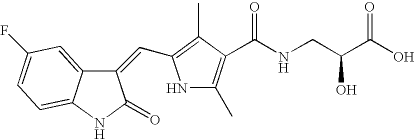

### Synthesis of (S)-methyl 3-amino-2-hydroxypropanoate hydrochloride

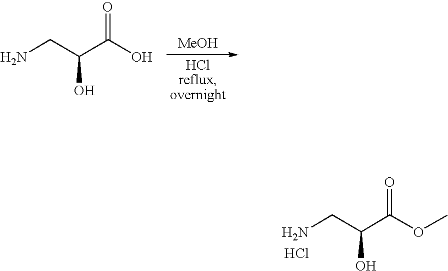

To the (S)-isoserine (921.6 mg, 8.77 mmol) in methanol (20 mL) was added concentrated HCl (0.5 mL), and the mixture was refluxed overnight. The mixture was cooled to 25° C. and the solvent was removed under reduced pressure. The crude material was dried and used directly in the next step.

### Synthesis of (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid methyl ester

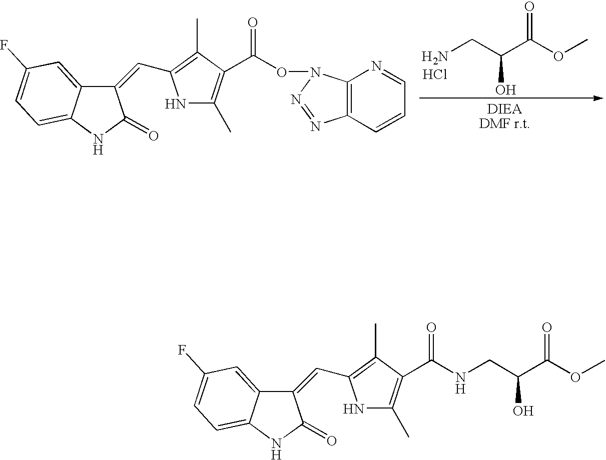

To (S)-methyl 3-amino-2-hydroxypropanoate hydrochloride (172.3 mg, 1.11 mmol) DMF solution was added DIEA (0.48 mL, 2.76 mmol) and the mixture was stirred at 25° C. for 20 minutes. (Z)-3H-[1,2,3]triazolo[4,5-b]pyridin-3-yl 5-((5-fluoro-2-oxoindolin-3-ylidene)methyl)-2,4-dimethyl-1H-pyrrole-3-carboxylate (174.8 mg, 0.418 mmol) was added, and the mixture was stirred at 25° C. for the completion. The solvent was removed under reduced pressure to afford (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid methyl ester (quantitative yield). The product was used in the next step with no purification. LC-ESIMS observed [M+H]⁺ 401.98 (calculated for C₂₀H₂₀FN₃O₅ 401.15).

### Synthesis of (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid

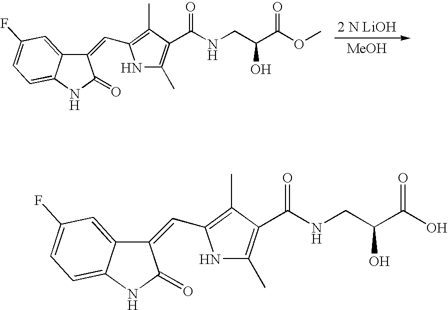

(S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid methyl ester (167 mg, 0.418 mmol) and LiOH.H₂O (36 mg, 0.86 mmol) and methanol/water (10 ml/2 mL) was stirred at 25° C. overnight. Most of the solvent was removed under reduced pressure and excess 1N HCl was added to acidify the mixture. The orange solid was collected by filtration and washed with cold methanol to afford (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid (yield 88%). LCESIMS observed [M+H]⁺ 387.96 (calculated for C₁₉H₁₈FN₃O₅ 387.12); ¹H NMR (400 MHz, DMSO-d₆) δ 13.91 (s, 1H), 10.89 (s, 1H), 7.75 (dd, J=9.6 Hz, 2.4 Hz, 1H), 7.70 (s, 1H), 7.57 (t, J=6.2 Hz, 1H), 6.92 (td, J=9.2 Hz, 2.4 Hz, 1H), 6.85-6.82 (m, 1H), 4.17-4.14 (m, 1H), 3.64 (s, 1H), 3.55-3.49 (m, 1H), 3.45-3.39 (m, 1H), 2.43 (s, 3H), 2.41 (s, 3H).

Examples 13-17: The general procedure for the synthesis of amides: An amine (1.2 equiv) was added to a suspension of the (Z)-3H-[1,2,3]-triazolo[4,5-b]pyridin-3-yl 5-((5-fluoro-2-oxoindolin-3-ylidene)methyl)-2,4-dimethyl-1H-pyrrole-3-carboxylate (1.0 eq) in DMF. The mixture was stirred at 25° C. for 2 h and LC/MS was applied to detect the completion of the reaction. The final solution was removed to get the crude solid, which was precipitated in 5% diethylamine/methanol, the solid was collected by filtration and washed with 5% diethylamine/methanol to afford the pure amide product, which was subsequently characterized by LC-MS and NMR spectroscopy.

### Example 13

### Synthesis of 5-[5-Fluoro-2-oxo-1,2-dihydro-indol-(3Z)-ylidenemethyl]-2,4-dimethyl-1H-pyrrole-3-carboxylic acid ((S)-2-dimethylcarbamoyl-2-hydroxy-ethyl)-amide

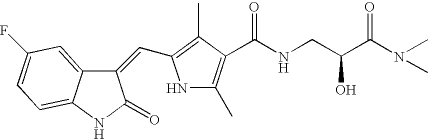

### Synthesis of (S)-3-(benzyloxycarbonyl)-2-hydroxypropanoic acid

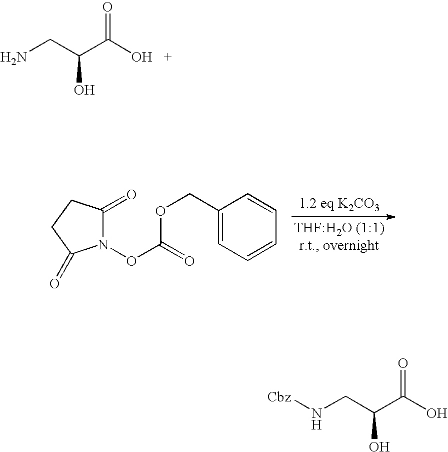

To the THF/water (50 mL/50 mL) solution of (S)-isoserine (2.429 g, 23.12 mmol) was added K₂CO₃ (3.834 g, 27.74 mmol) and N-(Benzyloxycarbonyloxy)-succinimide (5.76 g, 23.11 mmol). The reaction mixture was stirred at 25° C. overnight. The reaction mixture was concentrated and diluted with EtOAc and acidified with excess HCl. The aqueous layer was extracted with EtOAc, and the combined organic layers were washed with dilute HCl, water, brine and dried over sodium sulfate. The solvent was removed under reduced pressure to afford (S)-3-(benzyloxycarbonyl)-2-hydroxypropanoic acid (5.11 g, 92%), which was used in the next step with no further purification. LC-ESIMS observed [M+H]⁺ 239.91 (calculated for C₁₁H₁₃NO₅ 239.08).

### Synthesis of (S)-benzyl 3-(dimethylamino)-2-hydroxy-3-oxopropylcarbamate

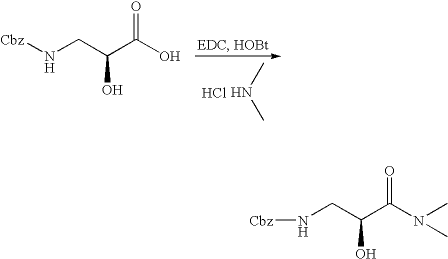

To (S)-3-(benzyloxycarbonyl)-2-hydroxypropanoic acid (377.8 mg, 1.58 mmol) in DMF (5 mL) was added dimethylamine hydrogen chloride (193.2 mg, 2.37 mmol) and DIEA (0.9 mL, 5.17 mmol). The mixture was then stirred for 5 min and EDC (454.3 mg, 2.37 mmol) and HOBt (320.3 mg, 2.37 mmol) were added. The reaction mixture was stirred at 25° C. overnight. DMF was removed under reduced pressure and the crude material was diluted with EtOAc and washed with saturated NaHCO₃. The aqueous layer was extracted twice with EtOAc and the combined organic layers were washed with water, 1N HCl and dried over NaSO₄. The solution was concentrated and the crude material was purified by flash chromatography with 0-20% MeOH/DCM to obtain the (S)-benzyl 3-(dimethylamino)-2-hydroxy-3-oxopropylcarbamate (349.2 mg, 83%). LC-ESIMS observed [M+H]⁺ 266.96 (calculated for C₁₃H₁₈N₂O₄ 266.13).

### Synthesis of (S)-3-amino-2-hydroxy-N,N-dimethylpropanamide

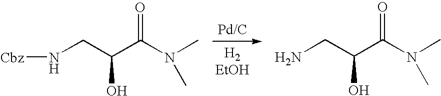

To the degassed (S)-benzyl 3-(dimethylamino)-2-hydroxy-3-oxopropylcarbamate (256.6 mg, 0.964 mmol) in ethanol (10 mL) was added Pd/C (10%, 30 mg) under argon protection, and then the mixture was degassed. The hydrogen balloon was used to provide the H₂ source. The reaction was stirred at 50° C. overnight. The mixture was filtered with Celite 521. The filtrate was evaporated to afford (S)-3-amino-2-hydroxy-N,N-dimethylpropanamide (125.2 mg, 98%). ¹H NMR (400 MHz, CDCl₃) δ 4.65 (t, J=5.4 Hz, 1H), 3.71-3.59 (m, 2H), 3.07 (s, 3H), 3.04 (s, 3H), 1.94 (broad s, 2H).

### Synthesis of 5-[5-Fluoro-2-oxo-1,2-dihydro-indol-(3Z)-ylidenemethyl]-2,4-dimethyl-1H-pyrrole-3-carboxylic acid ((S)-2-dimethylcarbamoyl-2-hydroxy-ethyl)-amide

The title compound was obtained following the general procedure for amide synthesis (79%). LC-ESIMS observed [M+H]⁺ 414.97 (calculated for C₂₁H₂₃FN₄O₄414.17); ¹H NMR (400 MHz, DMSO-d₆) δ 13.68 (s, 1H), 10.89 (s, 1H), 7.76 (dd, J=9.6 Hz, 2.4 Hz, 1H), 7.71 (s, 1H), 7.59 (t, J=6.2 Hz, 1H), 6.92 (td, J=9.2 Hz, 2.4 Hz, 1H), 6.85-6.82 (m, 1H), 5.04 (d, J=7.6 Hz, 1H), 4.53 (q, J=6.2 Hz, 1H), 3.47-3.41 (m, 1H), 3.36-3.30 (m, 1H), 3.08 (s, 3H), 2.85 (s, 3H), 2.43 (s, 3H), 2.40 (s, 3H).

### Example 14

### Synthesis of 5-[5-Fluoro-2-oxo-1,2-dihydro-indol-(3Z)-ylidenemethyl]-2,4-dimethyl-1H-pyrrole-3-carboxylic acid ((S)-2-hydroxy-3-morpholin-4-yl-3-oxo-propyl)-amide

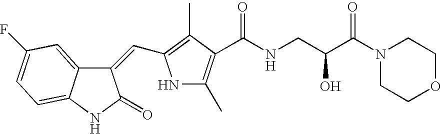

### Synthesis of (S)-benzyl 2-hydroxy-3-morpholino-3-oxopropylcarbamate

Similar method to synthesis of (S)-benzyl 3-(dimethylamino)-2-hydroxy-3-oxopropylcarbamate was applied and the title compound was obtained (yield 86%). LC-ESIMS observed [M+H]⁺ 408.96 (calculated for C₁₅H₂₀N₂O₅ 308.96).

### Synthesis of (S)-3-amino-2-hydroxy-1-morpholinopropan-1-one

Similar method to synthesis of (S)-3-amino-2-hydroxy-N,N-dimethylpropanamide was applied and the title compound was obtained (yield 94%). ¹H NMR (400 MHz, CDCl₃) δ 4.36-4.34 (m, 1H), 3.75-3.54 (m, 8H), 3.50 (d, J=4.0 Hz, 1H), 2.96-2.79 (m, 2H), 1.94 (broad s, 2H).

### Synthesis of 5-[5-Fluoro-2-oxo-1,2-dihydro-indol-(3Z)-ylidenemethyl]-2,4-dimethyl-1H-pyrrole-3-carboxylic acid ((S)-2-hydroxy-3-morpholin-4-yl-3-oxo-propyl)-amide

The title compound was obtained following the general procedure for amide synthesis (75%). LC-ESIMS observed [M+H]⁺ 457.01 (calculated for C₂₃H₂₅FN₄O₅ 456.18); ¹H NMR (400 MHz, DMSO-d₆) δ 13.68 (s, 1H), 10.89 (s, 1H), 7.76 (dd, J=9.6 Hz, 2.4 Hz, 1H), 7.71 (s, 1H), 7.59 (t, J=6.2 Hz, 1H), 6.92 (td, J=9.2 Hz, 2.4 Hz, 1H), 6.85-6.82 (m, 1H), 5.18 (d, J=8.8 Hz, 1H), 4.51 (q, J=6.0 Hz, 1H), 3.61-3.51 (m, 6H), 3.49-3.36 (m, 4H), 2.43 (s, 3H), 2.41 (s, 3H).

### Example 15

### Synthesis of 5-[5-Fluoro-2-oxo-1,2-dihydro-indol-(3Z)-ylidenemethyl]-2,4-dimethyl-1H-pyrrole-3-carboxylic acid ((R)-2-dimethylcarbamoyl-2-hydroxy-ethyl)-amide

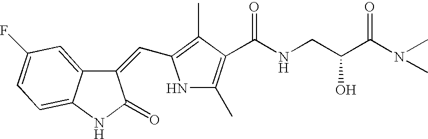

### Synthesis of (R)-methyl 3-azido-2-hydroxypropanoate

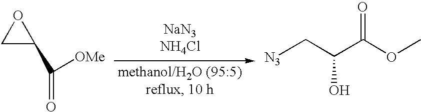

Sodium azide (5.487 g, 84.39 mmol) and ammonium chloride (2.257 g, 42.2 mmol) were added to a solution of methyl (2R)-glycidate (2.872 g, 28.13 mmol) in methanol (40 mL) and water (2 mL). After refluxing for 10 h, methanol was evaporated. The mixture was diluted in CHCl₃, washed with 1N HCl (5 mL) and extracted. After drying over sodium sulfate, the organic phase was concentrated and purified by flash chromatography to give the (R)-methyl 3-azido-2-hydroxypropanoate (2.82 g, 69%). ¹H NMR (400 MHz, CDCl₃) δ 4.39-4.36 (m, 1H), 3.84 (s, 3H), 3.67-3.48 (m, 2H), 3.18 (d, J=4.0 Hz, 1H).

### Synthesis of (R)-3-azido-2-hydroxypropanoic acid

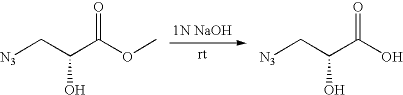

To a solution of (R)-methyl 3-azido-2-hydroxypropanoate (7.3 g, 50.3 mmol) in MeOH (150 mL) at 0° C. was added 1N NaOH (65 mL, 65 mmol). After being stirred at room temperature for 1 h, the mixture was acidified by 1N HCl and extracted with EtOAc. The organic layers were dried over sodium sulfate and concentrated in vacuo to give the acid as a white solid. The compound was used in the next step with no further purification.

### Synthesis of (R)-3-azido-2-hydroxy-N,N-dimethylpropanamide

Similar method to synthesis of (S)-benzyl 3-(dimethylamino)-2-hydroxy-3-oxopropylcarbamate was applied and the title compound was obtained (yield 93%). ¹H NMR (400 MHz, CDCl₃) δ 4.39-4.36 (m, 1H), 3.67-3.48 (m, 2H), 3.18 (d, J=4.0 Hz, 1H), 3.08 (s, 3H), 3.04 (s, 3H).

### Synthesis of (R)-3-amino-2-hydroxy-N,N-dimethylpropanamide

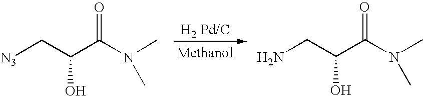

To the degassed (R)-3-azido-2-hydroxy-N,N-dimethylpropanamide (8.37 g, 46.6 mmol) in ethanol (150 mL) was added Pd/C (10%, 837 mg) under argon protection, and then the mixture was degassed. A hydrogen balloon was used to provide an H₂ source. The reaction was stirred at 25° C. for 2 h, and TLC was applied to detect the completion of the reaction. The mixture was filtered with Celite 521. The filtrate was evaporated to afford the desired compound (5.38 g, 87%). ¹H NMR (400 MHz, CDCl₃) δ 4.65 (t, J=5.4 Hz, 1H), 3.71-3.59 (m, 2H), 3.07 (s, 3H), 3.04 (s, 3H).

### Synthesis of 5-[5-Fluoro-2-oxo-1,2-dihydro-indol-(3Z)-ylidenemethyl]-2,4-dimethyl-1H-pyrrole-3-carboxylic acid ((R)-2-dimethylcarbamoyl-2-hydroxy-ethyl)-amide

The title compound was obtained following the general procedure for amide synthesis (yield 85%), LC-ESIMS observed [M+H]⁺ 414.97 (calculated for C₂₁H₂₃FN₄O₄414.17); ¹H NMR (400 MHz, DMSO-d₆) δ 13.67 (s, 1H), 10.89 (s, 1H), 7.76 (dd, J=9.6 Hz, 2.4 Hz, 1H), 7.71 (s, 1H), 7.59 (t, J=6.2 Hz, 1H), 6.92 (td, J=9.2 Hz, 2.4 Hz, 1H), 6.85-6.82 (m, 1H), 5.04 (d, J=7.6 Hz, 1H), 4.53 (q, J=6.2 Hz, 1H), 3.47-3.41 (m, 1H), 3.36-3.30 (m, 1H), 3.08 (s, 3H), 2.85 (s, 3H), 2.43 (s, 3H), 2.40 (s, 3H).

### Example 16

### Synthesis of 5-[5-Fluoro-2-oxo-1,2-dihydro-indol-(3Z)-ylidenemethyl]-2,4-dimethyl-1H-pyrrole-3-carboxylic acid ((R)-2-hydroxy-3-morpholin-4-yl-3-oxo-propyl)-amide

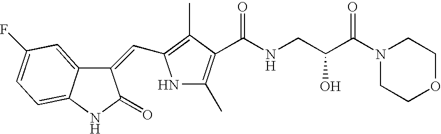

### Synthesis of (R)-3-azido-2-hydroxy-1-morpholinopropan-1-one

Similar method to synthesis of (S)-benzyl 3-(dimethylamino)-2-hydroxy-3-oxopropylcarbamate was applied and the title compound was obtained (yield 90%), ¹H NMR (400 MHz, CDCl₃) δ 4.55 (t, J=5.2 Hz, 1H), 3.71-3.60 (m, 6H), 3.48-3.41 (m, 3H), 3.40-3.35 (m, 2H).

### Synthesis of (R)-3-amino-2-hydroxy-1-morpholinopropan-1-one

A similar method to synthesis of (R)-3-amino-2-hydroxy-N,N-dimethylpropanamide was used and the title compound was obtained in high yield (yield 95%). ¹H NMR (400 MHz, CDCl₃) δ 4.36-4.34 (m, 1H), 3.75-3.54 (m, 8H), 3.50 (d, J=4.0 Hz, 1H), 2.96-2.79 (m, 2H), 1.94 (broad s, 2H).

### Synthesis of 5-[5-Fluoro-2-oxo-1,2-dihydro-indol-(3Z)-ylidenemethyl]-2,4-dimethyl-1H-pyrrole-3-carboxylic acid ((R)-2-hydroxy-3-morpholin-4-yl-3-oxo-propyl)-amide

The title compound was obtained following the general procedure for amide synthesis (yield 75%). LC-ESIMS observed [M+H]⁺ 457.01 (calculated for C₂₃H₂₅FN₄O₅ 456.18); ¹H NMR (400 MHz, DMSO-d₆) δ 13.68 (s, 1H), 10.89 (s, 1H), 7.76 (dd, J=9.6 Hz, 2.4 Hz, 1H), 7.71 (s, 1H), 7.59 (t, J=6.2 Hz, 1H), 6.92 (td, J=9.2 Hz, 2.4 Hz, 1H), 6.85-6.82 (m, 1H), 5.18 (d, J=6.4 Hz, 1H), 4.54-4.49 (m, 1H), 3.61-3.51 (m, 6H), 3.49-3.36 (m, 4H), 2.43 (s, 3H), 2.41 (s, 3H).

### Example 17

### Synthesis of 5-[5-Fluoro-2-oxo-1,2-dihydro-indol-(3Z)-ylidenemethyl]-2,4-dimethyl-1H-pyrrole-3-carboxylic acid ((R)-2-hydroxy-2-methylcarbamoyl-ethyl)-amide

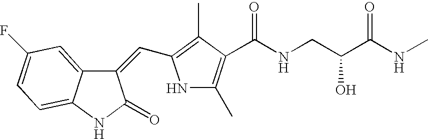

### Synthesis of (R)-3-azido-2-hydroxy-N-methylpropanamide

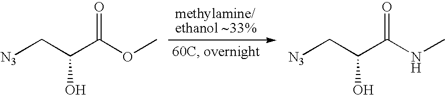

(R)-methyl 3-azido-2-hydroxypropanoate (505.4 mg, 3.48 mmol) and methylamine ethanol solution (15 mL) was sealed and stirred at 60° C. oil bath overnight. TLC analysis was applied to detect the reaction completion. The solvent was removed and the crude was purified by flash chromatography (0˜20% Methanol/DCM) to afford (R)-3-azido-2-hydroxy-N-methylpropanamide (385.2 mg, yield 77%), ¹H NMR (400 MHz, CDCl₃) δ 6.90-6.70 (broad s, 1H), 4.28-4.24 (m, 1H), 3.69-3.57 (m, 3H), 2.87 (d, J=5.6 Hz, 3H).

### Synthesis of (R)-3-amino-2-hydroxy-N-methylpropanamide

Similar method to synthesis of (R)-3-amino-2-hydroxy-N,N-dimethylpropanamide was used and the title compound was obtained (yield 98%). ¹H NMR (400 MHz, CDCl₃) δ 7.05 (broad s, 1H), 3.97 (t, J=5.6 Hz, 1H), 3.12-2.96 (m, 2H), 2.85 (d, J=5.2 Hz, 3H), 1.90 (broad, 2H).

### Synthesis of 5-[5-Fluoro-2-oxo-1,2-dihydro-indol-(3Z)-ylidenemethyl]-2,4-dimethyl-1H-pyrrole-3-carboxylic acid ((R)-2-hydroxy-2-methylcarbamoyl-ethyl)-amide

The title compound was obtained following the general procedure for amide synthesis (yield 86%), LC-ESIMS observed [M+H]⁺ 400.96 (calculated for C₂₀H₂₁FN₄O₄400.15); ¹H NMR (400 MHz, DMSO-d₆) δ 13.69 (s, 1H), 10.89 (s, 1H), 7.87 (d, J=4.8 Hz, 1H), 7.76 (dd, J=9.6 Hz, 2.4 Hz, 1H), 7.71 (s, 1H), 7.52 (t, J=5.6 Hz, 1H), 6.95-6.90 (m, 1H), 6.85-6.82 (m, 1H), 5.83 (d, J=5.2 Hz, 1H), 4.07-4.03 (m, 1H), 3.57-3.51 (m, 1H), 3.37-3.30 (m, 1H), 2.62 (d, J=4.4 Hz, 3H) 2.45 (s, 3H), 2.42 (s, 3H).
Examples 18-217: Still further amide examples are shown in the following table:

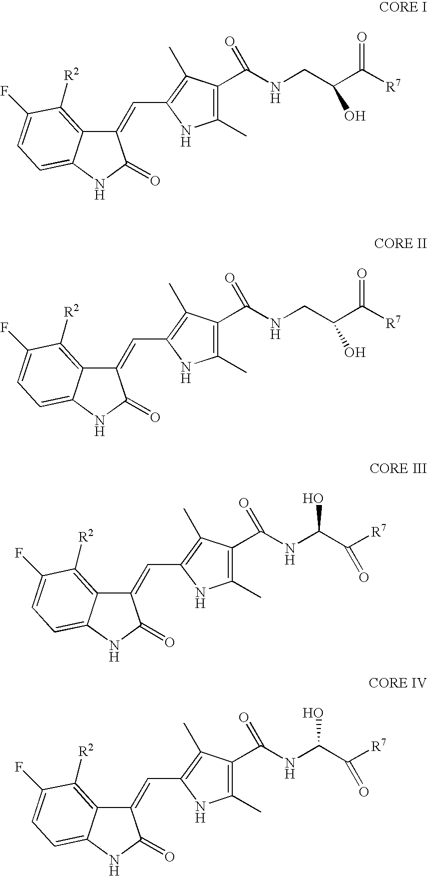

In the above core structures, R² is selected from the group consisting of hydrogen and fluoro; and R⁷ is selected from the group consisting of hydroxyl or radicals represented by the following structures:

| Ex# | Core | R⁷ |
|---|---|---|
| 18 | I | a |
| 19 | I | b |
| 20 | I | c |
| 21 | I | d |
| 22 | I | e |
| 23 | I | f |
| 24 | I | g |
| 25 | I | h |
| 26 | I | i |
| 27 | I | j |
| 28 | I | k |
| 29 | I | l |
| 30 | I | m |
| 31 | I | n |
| 32 | I | o |
| 33 | I | p |
| 34 | I | q |
| 35 | I | r |
| 36 | I | s |
| 37 | I | t |
| 38 | I | u |
| 39 | I | V |
| 40 | I | w |
| 41 | I | x |
| 42 | I | y |
| 43 | I | z |
| 44 | I | aa |
| 45 | I | ab |
| 46 | I | ac |
| 47 | I | ad |
| 48 | I | ae |
| 49 | I | af |
| 50 | I | ag |
| 51 | I | ah |
| 52 | I | ai |
| 53 | I | aj |
| 54 | I | ak |
| 55 | I | al |
| 56 | I | am |
| 57 | I | an |
| 58 | I | ao |
| 59 | I | ap |
| 60 | I | aq |
| 61 | I | ar |
| 62 | I | as |
| 63 | I | at |
| 64 | I | au |
| 65 | I | av |
| 66 | I | aw |
| 67 | I | ax |
| 68 | II | a |
| 69 | II | b |
| 70 | II | c |
| 71 | II | d |
| 72 | II | e |
| 73 | II | f |
| 74 | II | g |
| 75 | II | h |
| 76 | II | i |
| 77 | II | j |
| 78 | II | k |
| 79 | II | l |
| 80 | II | m |
| 81 | II | n |
| 82 | II | o |
| 83 | II | p |
| 84 | II | q |
| 85 | II | r |
| 86 | II | s |
| 87 | II | t |
| 88 | II | u |
| 89 | II | v |
| 90 | II | w |
| 91 | II | x |
| 92 | II | y |
| 93 | II | z |
| 94 | II | aa |
| 95 | II | ab |
| 96 | II | ac |
| 97 | II | ad |
| 98 | II | ae |
| 99 | II | af |
| 90 | II | ag |
| 100 | II | ah |
| 102 | II | ai |
| 103 | II | aj |
| 104 | II | ak |
| 105 | II | al |
| 106 | II | am |
| 107 | II | an |
| 108 | II | ao |
| 109 | II | ap |
| 110 | II | aq |
| 111 | II | ar |
| 112 | II | as |
| 113 | II | at |
| 114 | II | au |
| 115 | II | av |
| 116 | II | aw |
| 117 | II | ax |
| 118 | III | a |
| 119 | III | b |
| 120 | III | c |
| 121 | III | d |
| 122 | III | e |
| 123 | III | f |
| 124 | III | g |
| 125 | III | h |
| 126 | III | i |
| 127 | III | j |
| 128 | III | k |
| 129 | III | l |
| 130 | III | m |
| 131 | III | n |
| 132 | III | o |
| 133 | III | p |
| 134 | III | q |
| 135 | III | r |
| 136 | III | s |
| 137 | III | t |
| 138 | III | u |
| 139 | III | v |
| 140 | III | w |
| 141 | III | x |
| 142 | III | y |
| 143 | III | z |
| 144 | III | aa |
| 145 | III | ab |
| 146 | III | ac |
| 147 | III | ad |
| 148 | III | ae |
| 149 | III | af |
| 150 | III | ag |
| 151 | III | ah |
| 152 | III | ai |
| 153 | III | aj |
| 154 | III | ak |
| 155 | III | al |
| 156 | III | am |
| 157 | III | an |
| 158 | III | ao |
| 159 | III | ap |
| 160 | III | aq |
| 161 | III | ar |
| 162 | III | as |
| 163 | III | at |
| 164 | III | au |
| 165 | III | av |
| 166 | III | aw |
| 167 | III | ax |
| 168 | IV | a |
| 169 | IV | b |
| 170 | IV | c |
| 171 | IV | d |
| 172 | IV | e |
| 173 | IV | f |
| 174 | IV | g |
| 175 | IV | h |
| 176 | IV | i |
| 177 | IV | j |
| 178 | IV | k |
| 179 | IV | l |
| 180 | IV | m |
| 181 | IV | n |
| 182 | IV | o |
| 183 | IV | p |
| 184 | IV | q |
| 185 | IV | r |
| 186 | IV | s |
| 187 | IV | t |
| 188 | IV | u |
| 189 | IV | v |
| 190 | IV | w |
| 191 | IV | x |
| 192 | IV | y |
| 193 | IV | z |
| 194 | V | aa |
| 195 | V | ab |
| 196 | V | ac |
| 197 | V | ad |
| 198 | V | ae |
| 199 | V | af |
| 200 | V | ag |
| 201 | V | ah |
| 202 | V | ai |
| 203 | V | aj |
| 204 | V | ak |
| 205 | V | ai |
| 206 | V | am |
| 207 | V | an |
| 208 | V | ao |
| 209 | V | ap |
| 210 | V | aq |
| 211 | V | ar |
| 212 | V | as |
| 213 | V | at |
| 214 | V | au |
| 215 | V | av |
| 216 | V | aw |
| 217 | V | ax |

In the above table, R⁷ is selected from the following radicals:

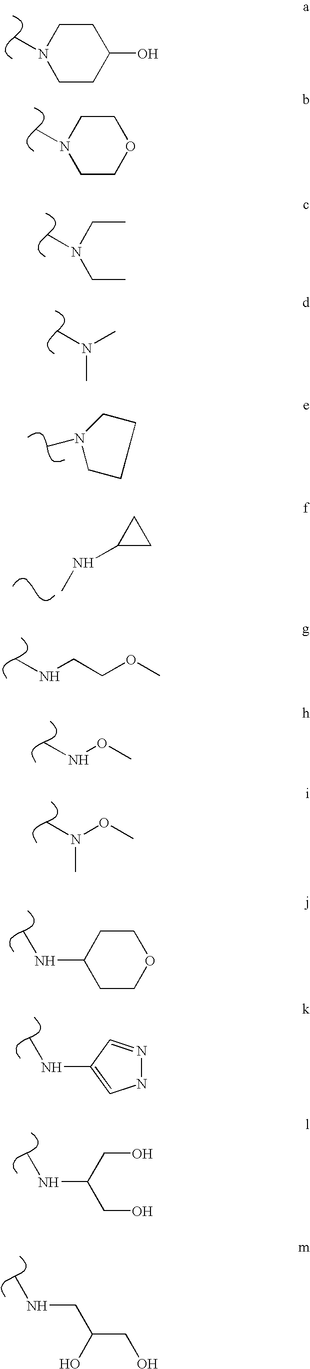

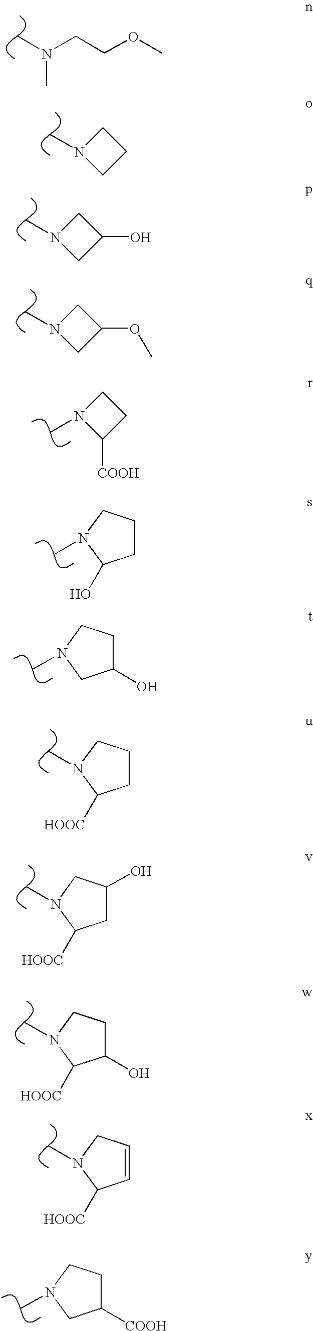

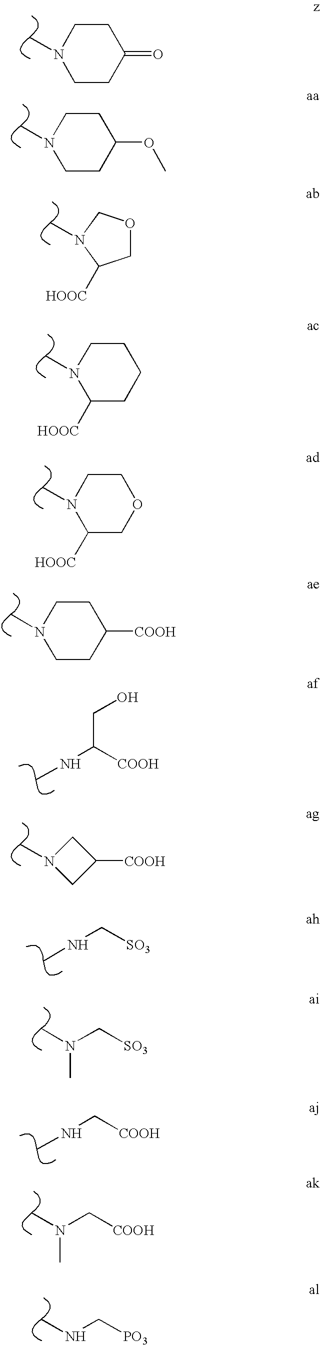

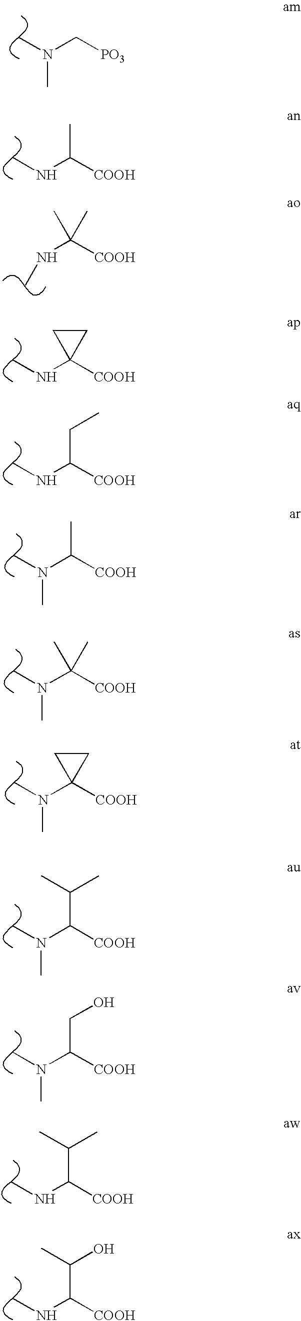

These amide examples 18-217 can be made by those skilled in the art following the above procedure and/or known procedures.

### VEGFR Biochemical Assay

The compounds were assayed for biochemical activity by Upstate Ltd at Dundee, United Kingdom, according to the following procedure. In a final reaction volume of 25 μl, KDR (h) (5-10 mU) is incubated with 8 mM MOPS pH 7.0, 0.2 mM EDTA, 0.33 mg/ml myelin basic protein, 10 mM MgAcetate and [γ-³³P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### Cellular Assay: HUVEC: VEGF Induced Proliferation

The compounds were assayed for cellular activity in the VEGF induced proliferation of HUVEC cells. HUVEC cells (Cambrex, CC-2517) were maintained in EGM (Cambrex, CC-3124) at 37° C. and 5% CO₂. HUVEC cells were plated at a density 5000 cells/well (96 well plate) in EGM. Following cell attachment (1 hour) the EGM-medium was replaced by EBM (Cambrex, CC-3129)+0.1% FBS (ATTC, 30-2020) and the cells were incubated for 20 hours at 37° C. The medium was replaced by EBM+1% FBS, the compounds were serial diluted in DMSO and added to the cells to a final concentration of 0-5,000 nM and 1% DMSO. Following a 1 hour pre-incubation at 37° C. cells were stimulated with 10 ng/ml VEGF (Sigma, V7259) and incubated for 45 hours at 37° C. Cell proliferation was measured by BrdU DNA incorporation for 4 hours and BrdU label was quantitated by ELISA (Roche kit, 16472229) using 1M H₂SO₄ to stop the reaction. Absorbance was measured at 450 nm using a reference wavelength at 690 nm.

### DETAILED DESCRIPTION OF FIGURES

FIG. 1 is a scheme showing the synthesis of the acid 1-3 and the corresponding amides, 1-4. The starting carboxylic acid is synthesized according to the supplemental material of Sun, L.; et al., J. Med. Chem. 2003, 46, 1116-1119. The intermediate, 1-2, is formed by reaction of the acid with HATU in the presence of 3 equivalents of Hunig's base, or di-isopropyl ethylamine (DIEA). A solid precipitated after 15 minutes and the solid was isolated and characterized. This was then reacted with 1.5 equivalents of methyl (2S)-4-amino-2-hydroxybutyrate in DMF and 3 equivalents of Hunig's base. The methyl ester was hydrolyzed with 5 equivalents of KOH in water. Acidifying the reaction mixture enabled the isolation of the free acid, 1-3. This acid was then reacted with HATU in the presence of 3 equivalents of DIEA in DMF. An amine (2 equivalents) was added and after reacting for 2 hours, the amide was isolated by preparative HPLC.

FIG. 2 is a scheme showing the synthesis of the amide series, 2-3. The activated acid, 1-2 is reacted with methyl 3-amino-2-hydroxypropionate hydrochloride in the presence of 3 equivalents of base (DIEA) in DMF. After stirring for 2 h at room temperature, KOH, 5 equivalents, in water was added and stirring continued until ester hydrolysis was complete. The acid was isolated after acidification of the reaction mixture. The free acid was then added to HATU (1.05 equivalent), DIEA (5 equivalents), and an amine (2 equivalents) in DMF. The mixture was stirred for 2 h at room temperature and the mixture was acidified. The pure product was isolated by preparative HPLC.

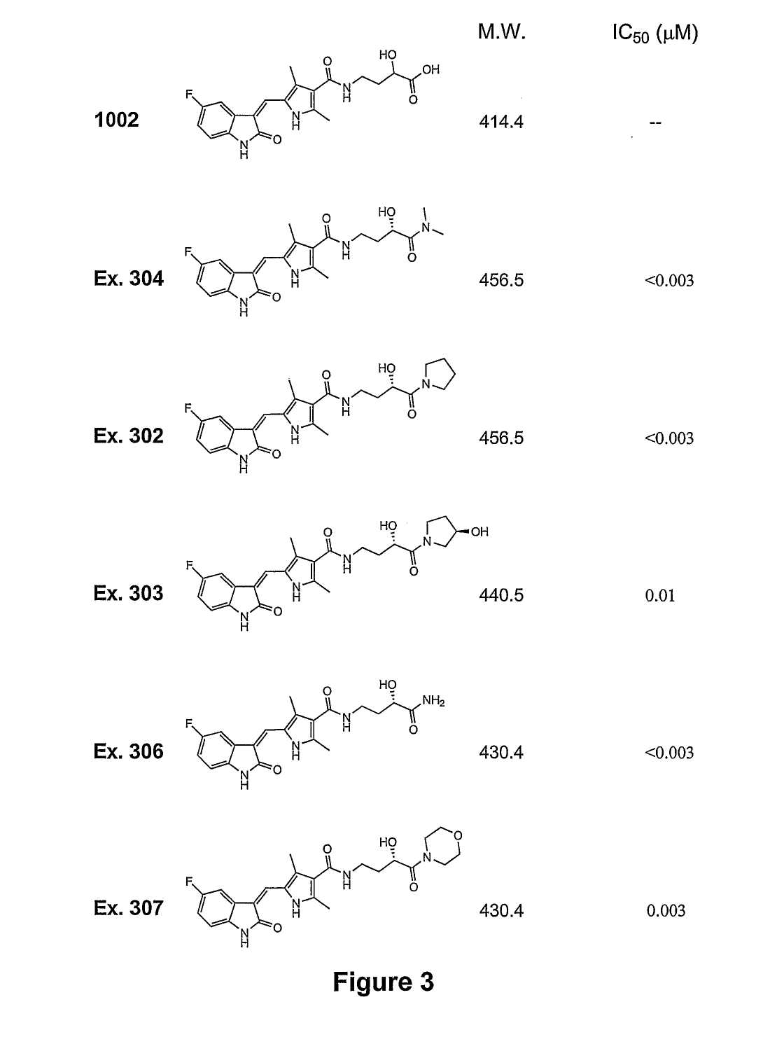

FIG. 3 shows example compounds and some of their activities against KDR. The units of IC₅₀ is in μM.

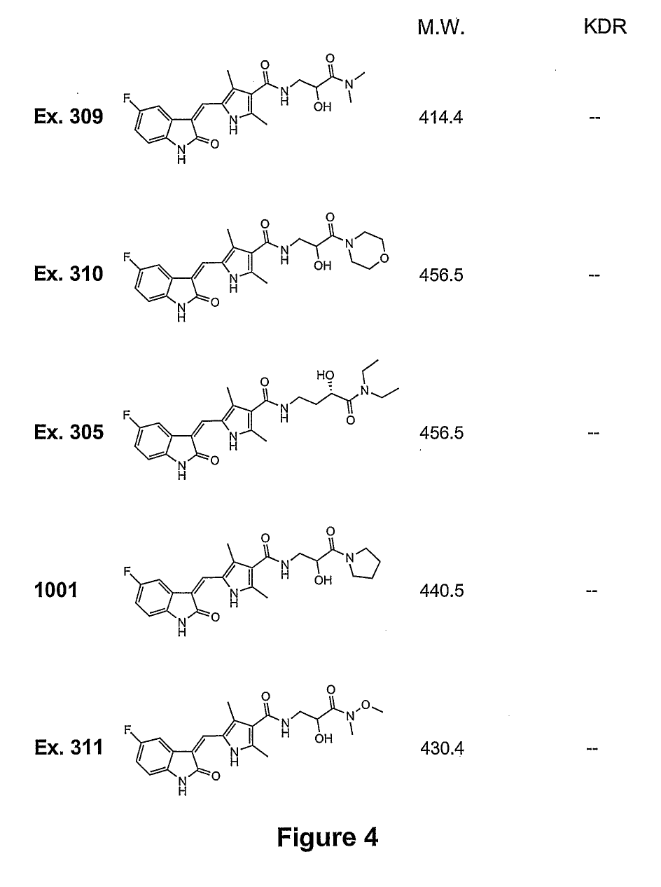

FIG. 4 shows additional compounds that were tested for activity.

What is claimed is:

1. A compound represented by Formula (I): 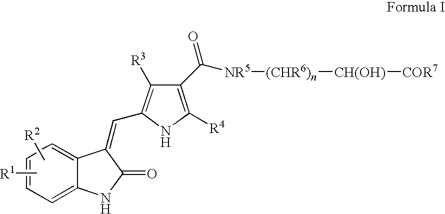
wherein:
R¹ is selected from the group consisting of hydrogen, halo, (C1-C6) alkyl, (C3-C8) cycloalkyl, (C1-C6) haloalkyl, hydroxy, (C1-C6) alkoxy, amino, (C1-C6) alkylamino, amide, sulfonamide, cyano, substituted or unsubstituted (C6-C10) aryl;
R² is selected from the group consisting of hydrogen, halo, (C1-C6) alkyl, (C3-C8) cycloalkyl, (C1-C6) haloalkyl, hydroxy, (C1-C6) alkoxy, (C2-C8) alkoxyalkyl, amino, (C1-C6) alkylamino, (C6-C10) arylamino;
R³ is selected from the group consisting of hydrogen, (C1-C6) alkyl, (C6-C10) aryl, (C5-C10) heteroaryl, and amide;
R⁴, R⁵ and R⁶ are independently selected from the group consisting of hydrogen and (C1-C6) alkyl;
R⁷ is selected from the group consisting of hydroxy, (C1-C6) O-alkyl, (C3-C8) O-cycloalkyl, and NR⁸R⁹; where R⁸ and R⁹ are independently selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids; and
n is 1, 2, or 3;
or, a pharmaceutically acceptable salt, its tautomer, a pharmaceutically acceptable salt of its tautomer, or a prodrug thereof.

2. The compound, salt, tautomer, or prodrug according to claim 1 selected from the group represented by the following structures: 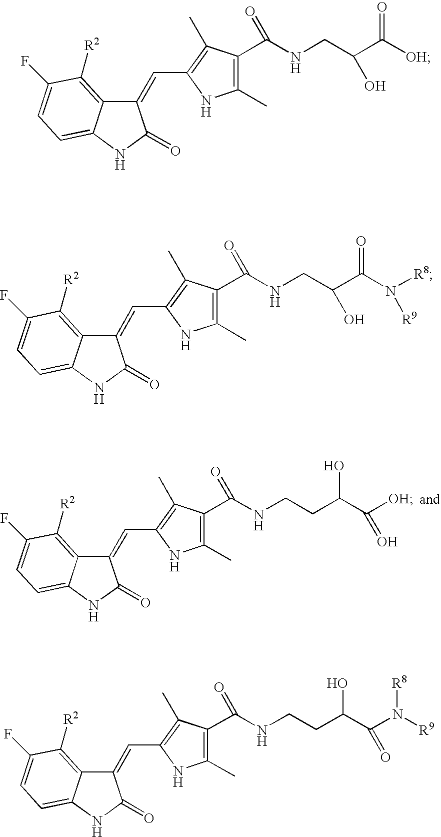
wherein R² is selected from the group consisting of hydrogen and fluoro.

3. The compound, salt, tautomer, or prodrug according to claim 1 represented by the following structure:

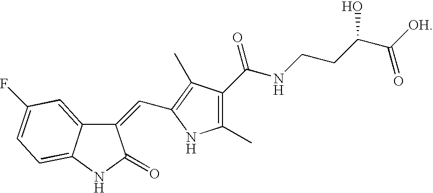

4. The compound, salt, tautomer, or prodrug according to claim 1 represented by Formula (II): 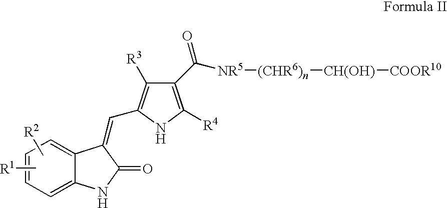
wherein R¹⁰ is selected from the group consisting of hydrogen, (C1-C6) alkyl, and (C3-C8) cycloalkyl.

5. The compound, salt, tautomer, or prodrug according to claim 4, wherein:
R¹ and R² are independently selected from the group consisting of hydrogen and fluoro;
R³ and R⁴ are methyl;
R⁵, R⁶, and R¹⁰ are hydrogen; and n is 1 or 2.

6. The compound, salt, tautomer, or prodrug according to claim 5 selected from the group consisting of:

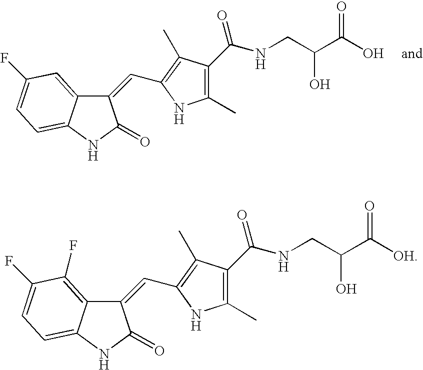

7. The compound, salt, tautomer, or prodrug according to claim 5 represented by the following structure:

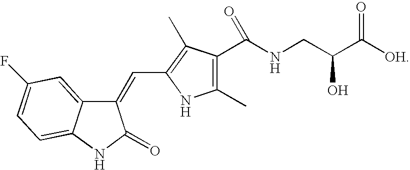

8. The compound, salt, tautomer, or prodrug represented by the following structure:

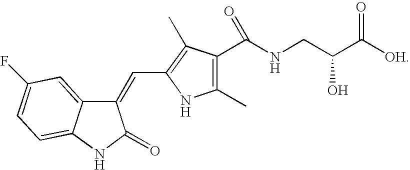

9. The compound, salt, tautomer, or prodrug according to claim 6 represented by the following structure:

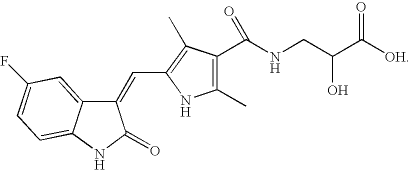

10. The compound, salt, tautomer, or prodrug according to claim 6 represented by the following structure:

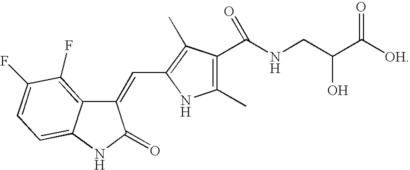

11. A compound, salt, tautomer, or prodrug according to claim 1 represented by Formula (III):

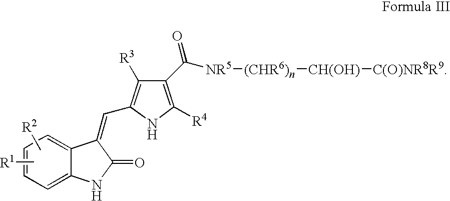

12. The compound, salt, tautomer, or prodrug of claim 11, wherein:
R¹ and R² are independently selected from the group consisting of hydrogen, halo, cyano;
R³, R⁴, R⁵ and R⁶ are independently hydrogen or (C1-C6))alkyl;
n is 1 or 2; and
R⁸ and R⁹ are selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids.

13. The compound, salt, tautomer, or prodrug according to claim 12 selected from the group represented by the following structures:

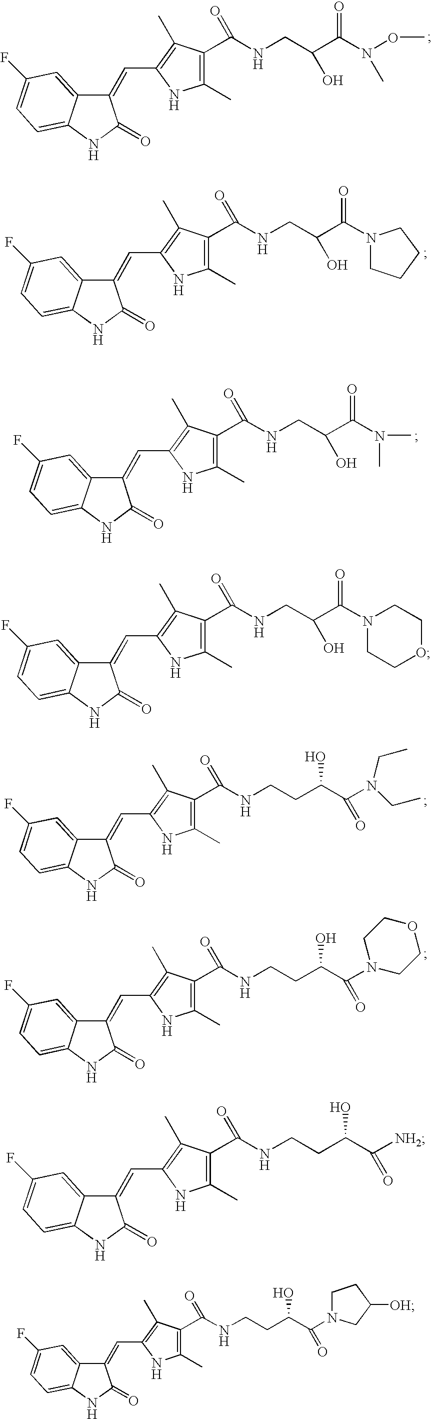

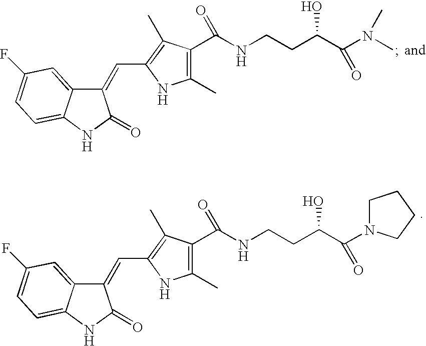

14. The compound, salt, tautomer, or prodrug according to claim 12 wherein n is 1.

15. The compound, salt, tautomer, or prodrug according to claim 13 represented by the following structures:

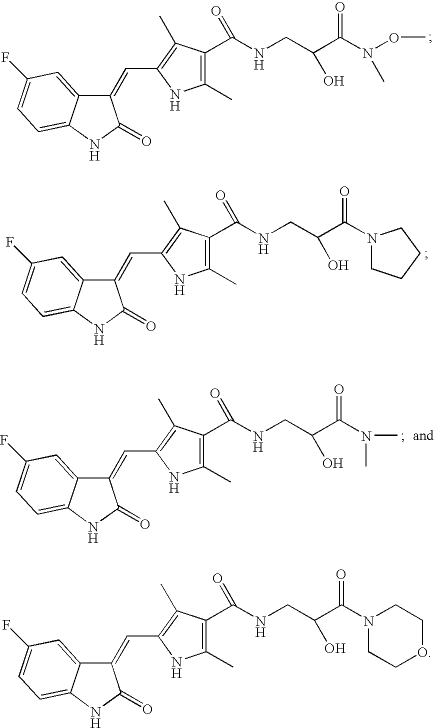

16. The compound, salt, tautomer, or prodrug according to claim 14 selected from the group represented by the following structures:

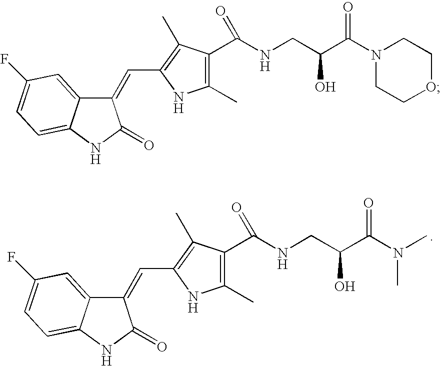

17. The compound, salt, tautomer, or prodrug according to claim 14 selected from the group represented by the following structures:

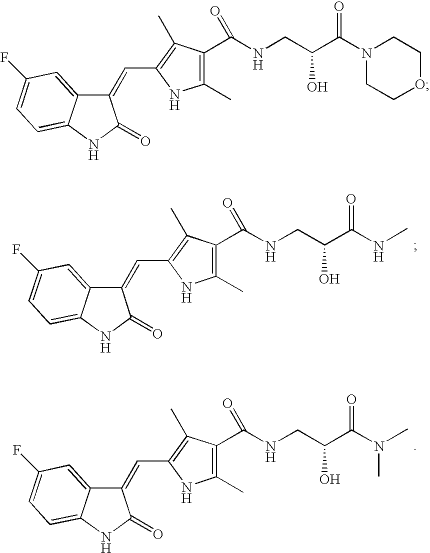

18. The compound, salt, tautomer, or prodrug represented by the following structure:

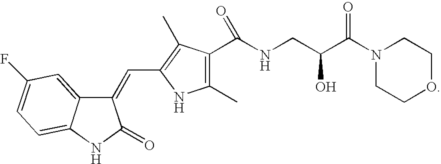

19. The compound, salt, tautomer, or prodrug represented by the following structure:

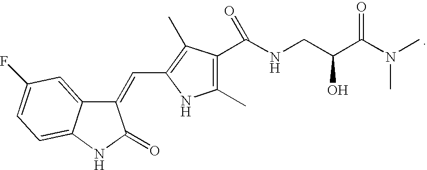

20. The compound, salt, tautomer, or prodrug represented by the following structure:

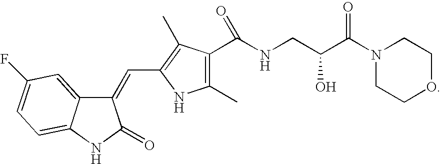

21. The compound, salt, tautomer, or prodrug according to claim 14 selected from the group represented by the following structures:

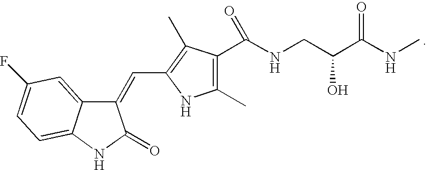

22. The compound, salt, tautomer, or prodrug according to claim 14 selected from the group represented by the following structures:

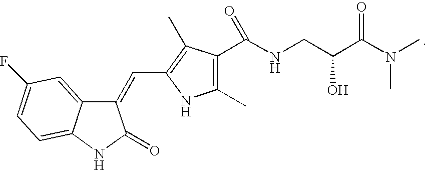

23. The compound, salt, tautomer, or prodrug according to claim 12 wherein n is 2.

24. The compound, salt, tautomer, or prodrug according to claim 23 represented by the following structures:

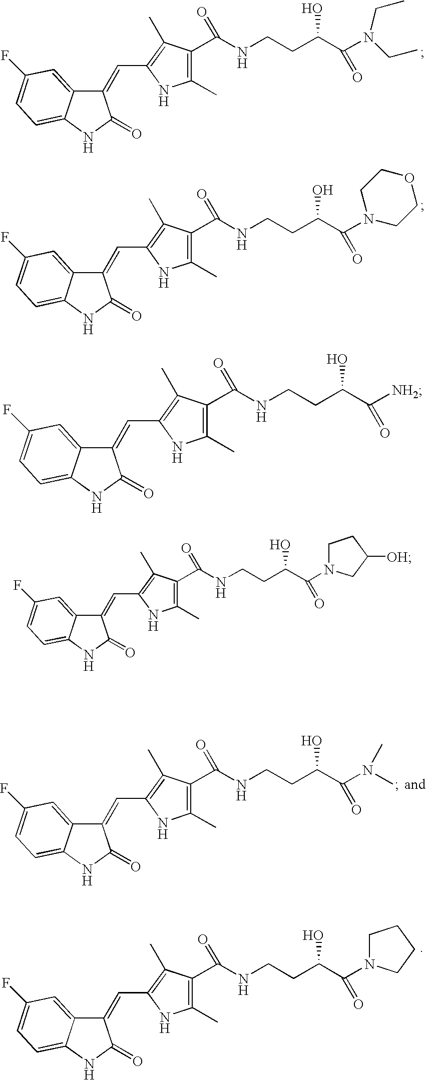

25. The compound, salt, tautomer, or prodrug according to claim 23 represented by the following structure:

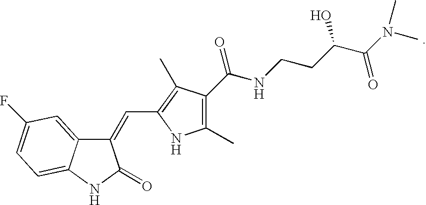

26. The compound, salt, tautomer, or prodrug according to claim 23 represented by the following structure:

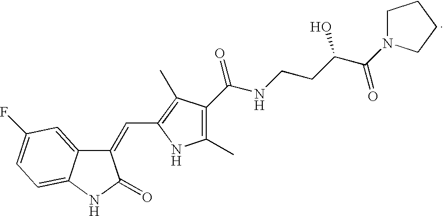

27. The compound, salt, tautomer, or prodrug according to claim 23 represented by the following structure:

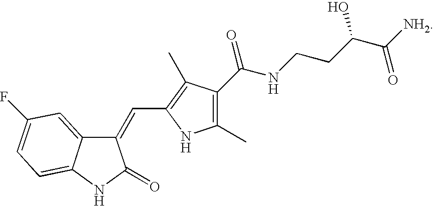

28. The compound, salt, tautomer, or prodrug according to claim 23 represented by the following structure:

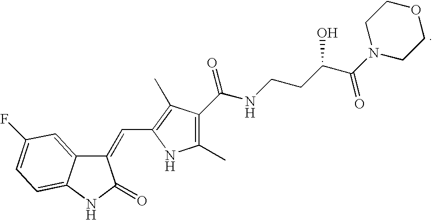

29. The compound, salt, tautomer, or prodrug according to claim 1 selected from the group represented by the following structures: 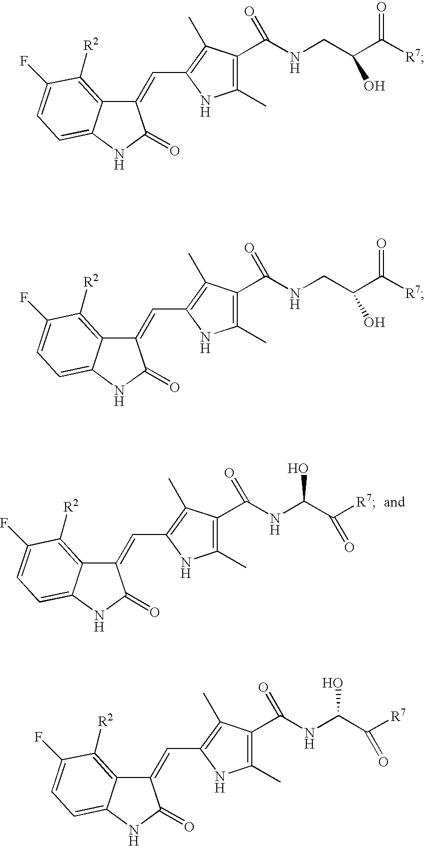
wherein:
R² is selected from the group consisting of hydrogen and fluoro; and
R⁷ is selected from the group consisting of hydroxyl or radicals represented by the following structures:

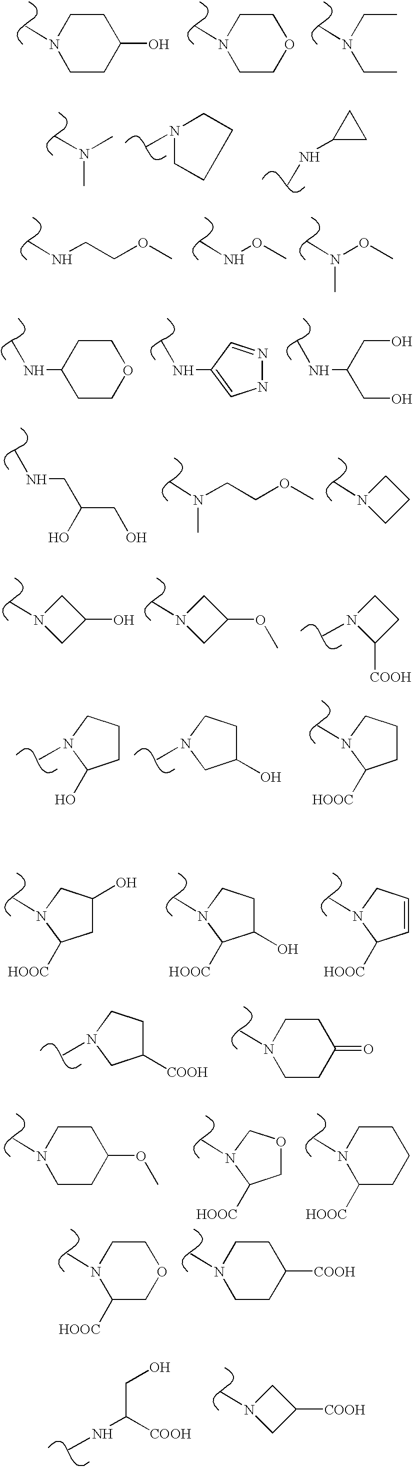

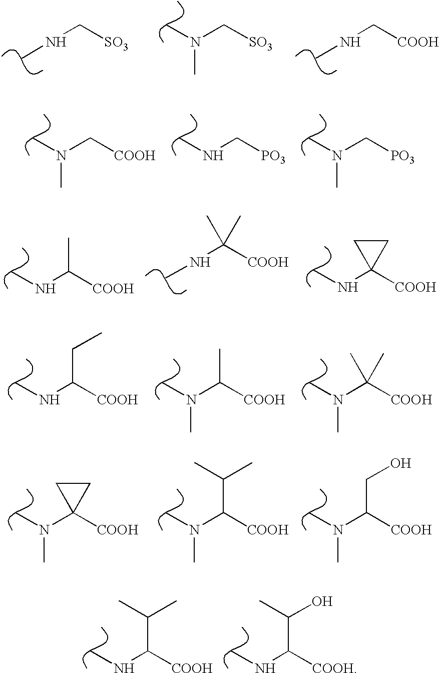

30. A method for the modulation of the catalytic activity of a protein kinase with a compound or salt of any one of claims 1-29.

31. The method of claim 30, wherein said protein kinase is selected from the group of receptors consisting of VEGF, PDGF, c-kit, Flt-3, Axl, and TrkA.

32. A process for synthesizing a pyrrolyl-indolinone having a chiral hydroxyl, the process comprising the following steps:
Step A: Converting a first intermediate to a second intermediate according to the following reaction: 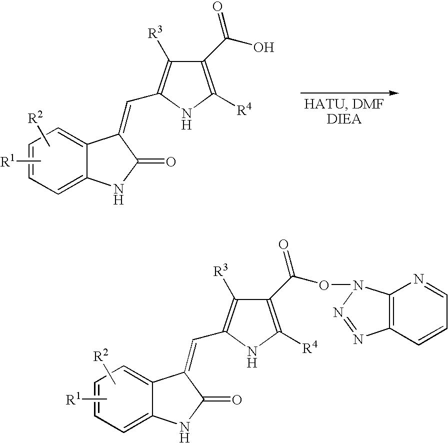
and then
Step B: Converting the second intermediate to the pyrrolyl-indolinone according to the following reaction: 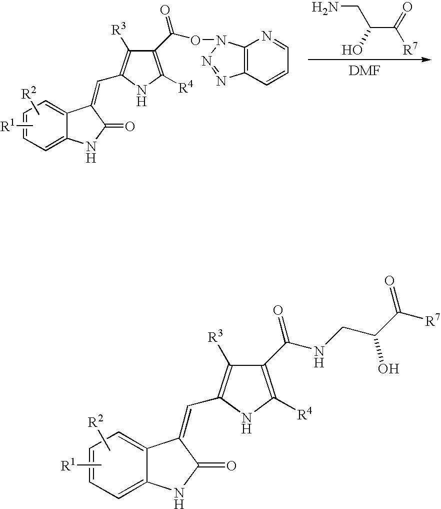
wherein:
R¹ is selected from the group consisting of hydrogen, halo, (C1-C6) alkyl, (C3-C8) cycloalkyl, (C1-C6) haloalkyl, hydroxy, (C1-C6) alkoxy, protected amino, protected (C1-C6) alkylamino, amide, sulfonamide, cyano, substituted or unsubstituted (C6-C10) aryl;
R² is selected from the group consisting of hydrogen, halo, (C1-C6) alkyl, (C3-C8) cycloalkyl, (C1-C6) haloalkyl, hydroxy, (C1-C6) alkoxy, (C2-C8) alkoxyalkyl, protected amino, protected (C1-C6) alkylamino, (C6-C10) arylamino;
R³ is selected from the group consisting of hydrogen, (C1-C6) alkyl, (C6-C10) aryl, (C5-C10) heteroaryl, and amide;
R⁴ is selected from the group consisting of hydrogen and (C1-C6) alkyl; and
R⁷ is selected from the group consisting of hydroxy, (C1-C6) O-alkyl, (C3-C8) O-cycloalkyl, and NR⁸R⁹; where R⁸ and R⁹ are independently selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids.
